(19) United States
(12) Patent Application Publication
(10) Pub. No.: US 2025/0347610 A1
(43) Pub. Date: Nov. 13, 2025

(54) FLOW CYTOMETER AND METHOD FOR SETTING WAVEFORM PARAMETERS OF SIGNAL FOR DRIVING DROPLET-GENERATING VIBRATION ELEMENT OF FLOW CYTOMETER

(71) Applicant: Sony Group Corporation, Tokyo (JP)

(72) Inventors: Tomoyuki Umetsu, Tokyo (JP); Shin Masuhara, Tokyo (JP)

(73) Assignee: Sony Group Corporation, Tokyo (JP)

(21) Appl. No.: 18/728,686

(22) PCT Filed: Jan. 13, 2023
(86) PCT No.: PCT/JP2023/000771

§ 371 Date: Jan. 17, 2025

(30) Foreign Application Priority Data
Jan. 21, 2022 (JP) 2022-007667

(51) Int. Cl. G01N 15/1404; G01N 15/1429; G01N 33/49

(52) U.S. Cl. CPC G01N 15/1404; G01N 15/1429; G01N 33/4915

(57) ABSTRACT

The present disclosure aims to provide a technique for stably controlling droplet formation.

The present disclosure provides a flow cytometer including a vibration control system that controls vibration of a vibration element that generates a droplet. The vibration control system is designed to drive the vibration element with a signal having a waveform in which a harmonic is superimposed on the waveform of a fundamental frequency, and the vibration control system sets a waveform parameter, on the basis of a change caused in a satellite droplet by a change in the waveform parameter of the harmonic.

### TECHNICAL FIELD

The present disclosure relates to a flow cytometer and a method for setting waveform parameters of a signal for driving a droplet-generating vibration element of the flow cytometer.

### BACKGROUND ART

A technology called flow cytometry is used for analyzing microparticles related to living bodies such as cells and microorganisms. This flow cytometry is an analysis method for analyzing and sorting microparticles by irradiating, with light, microparticles flowing so as to be included in a sheath flow fed into a flow channel formed in a flow cell, a microchip, or the like, and detecting fluorescence and scattered light emitted from each microparticle. A device that implements flow cytometry is called as a flow cytometer. Among flow cytometers, a device capable of performing particle sorting is also called a cell sorter.

To perform the particle sorting, a vibration element may be provided in part of the flow channel in which microparticles flow. The vibration element applies vibration to part of the flow channel, to continuously turn the fluid discharged from a discharge port of the flow channel into droplets. A predetermined electric charge is then applied to the droplets containing microparticles, the traveling direction of the droplets is changed by a deflection plate or the like on the basis of the electric charge, and only the target microparticles can be recovered into a predetermined container or a predetermined portion of a plate or the like.

Several techniques for stable droplet formation have been proposed so far. For example, Patent Document 1 discloses a microparticle analyzer that includes at least: a flow channel that allows passage of a fluid including a sample flow containing microparticles and a sheath flow that flows so as to surround the sample flow: a droplet forming unit that applies vibration to the fluid using a vibration element to form droplets in the fluid; a charging unit that applies an electric charge to the droplets containing the microparticles; an imaging unit that obtains a photograph of the phase at a certain time; and a control unit that controls the timing at which a droplet breaks off, on the basis of the photograph.

### CITATION LIST

### Patent Document

Patent Document 1: Japanese Translation of PCT International Application Publication No. 2021-517640

### SUMMARY OF THE INVENTION

### Problems to be Solved by the Invention

A technique for controlling droplet formation is one of important factors for increasing the accuracy of cell sorting. When the timing of break-off (droplet separation) at which a fluid discharged from a discharge port of a flow channel turns into droplets or the shape of the droplets is unstable, the amount of electric charge to be applied to the droplets also becomes unstable, which might adversely affect the accuracy of microparticle sorting. However, a plurality of factors such as the flow rate, the environmental conditions (temperature and/or humidity, and the like), and the particle size, for example, is involved in droplet formation, and therefore, it is difficult to control droplet formation. That is, droplet formation is likely to be affected by external disturbance.

In particular, experiments for the purpose of detecting or sorting rare cells are being often performed these days, and, in such experiments, the frequency to be used for droplet formation is becoming higher, and the time required for cell sorting is becoming longer. In such an experiment, more stable droplet formation and a higher extraction accuracy are required.

The present disclosure aims to provide a technique for stably controlling droplet formation.

### Solutions to Problems

The present disclosure provides
a flow cytometer including
a vibration control system that controls vibration of a vibration element that generates a droplet,
in which the vibration control system is designed to drive the vibration element with a signal having a waveform in which a harmonic is superimposed on the waveform of a fundamental frequency, and
the vibration control system sets a waveform parameter, on the basis of a change caused in a satellite droplet by a change in the waveform parameter of the harmonic.

The vibration control system may set the phase of the harmonic, the amplitude of the harmonic, or both the phase and the amplitude of the harmonic, on the basis of a satellite droplet in an image of a generated droplet.

The vibration control system may set the phase of the harmonic on the basis of a satellite droplet in an image of a generated droplet, and next set the amplitude of the harmonic on the basis of a satellite droplet in an image of a droplet generated when a harmonic having the set phase is adopted.

The vibration control system may set the phase of the harmonic, so as to change the timing at which a satellite droplet is recovered into a main droplet to an earlier timing.

The vibration control system may set the phase of the harmonic, on the basis of the change caused in a satellite droplet image by a change in the phase of the harmonic.

The vibration control system may
acquire a droplet image in each changed phase while changing the phase of the harmonic, and
determine the phase of the harmonic, on the basis of the acquired droplet images.

The vibration control system may change the phase of the harmonic so that the position at which a droplet is separated from the liquid column, and the distance between the position and the separated droplet are maintained.

The vibration control system may perform a classification process of classifying types of satellite droplets in each of the acquired droplet images, and a phase identification process of identifying an optimum phase on the basis of a classification result in the classification process.

In the classification process, a satellite droplet may be classified as a Fast satellite or a Slow satellite.

The vibration control system may set the amplitude of the harmonic, so as to separate a liquid portion forming a satellite droplet and a liquid portion forming a main droplet from the liquid column while the liquid portions are bonded to each other.

The vibration control system may determine the amplitude of the harmonic, on the basis of the change caused in the satellite droplet image by a change in the amplitude of the harmonic.

The vibration control system may
acquire a droplet image with each changed amplitude while changing the amplitude of the harmonic, and
determine the amplitude of the harmonic, on the basis of the acquired droplet images.

The vibration control system may change the amplitude of the harmonic so that the position at which a droplet is separated from the liquid column, and the distance between the position and the separated droplet are maintained.

The vibration control system may determine the amplitude of the harmonic, so as to separate a liquid portion forming a satellite droplet and a liquid portion forming a main droplet from the liquid column while the liquid portions remain bonded to each other.

The vibration control system may determine the amplitude of the harmonic, on the basis of a change in the state of bonding between the liquid portion forming a satellite droplet and the liquid portion forming a main droplet.

The vibration control system may determine the amplitude of the harmonic, on the basis of the width of the bonding portion between the liquid portion forming a satellite droplet and the liquid portion forming a main droplet.

The vibration control system may be designed to adjust the position at which a droplet is separated from the liquid column, and/or the distance between the position and the separated droplet.

The vibration control system may adjust the amplitude of the superimposed waveform, to adjust the position at which a droplet is separated from the liquid column and/or the distance between the position and the separated droplet.

The vibration control system may adjust the amplitude of the harmonic, to adjust the widths of the liquid portion forming a satellite droplet and the liquid portion forming a main droplet.

Also, the present disclosure provides
a method for setting a waveform parameter of a signal for driving a droplet-generating vibration element of a flow cytometer, the method including
a setting process of setting the waveform parameter of the signal for driving the droplet-generating vibration element,
in which the signal is a signal having a waveform in which a harmonic is superimposed on the waveform of a fundamental frequency, and
the setting process is performed on the basis of a change caused in a satellite droplet by a change in a waveform parameter of the harmonic.

### BRIEF DESCRIPTION OF DRAWINGS

FIG. 1A is a diagram illustrating an example configuration of a vibration control system.

FIG. 1B is a schematic diagram illustrating an example configuration of a flow cytometer.

FIG. 2 is a diagram for explaining types of satellite droplets.

FIG. 3 is a diagram illustrating an example configuration of a vibration element drive signal generation unit.

FIG. 4 is a diagram illustrating an example of a combined waveform formed by the fundamental wave and a harmonic.

FIG. 5 is an example of a flowchart of a waveform parameter setting process.

FIG. 6 is a diagram for explaining maintenance of the BOP and/or ΔBOP.

FIG. 7A is a diagram for explaining classification of Fast satellites and Slow satellites.

FIG. 7B is a diagram illustrating an example of a plot showing a slope representing changes change in the distance between a satellite droplet and a main droplet, and an example of a plot regarding the phase of the slope.

FIG. 8 is a diagram illustrating an example of droplet images acquired in step S106.

FIG. 9 is a diagram for explaining that the state of bonding between the liquid portion forming a satellite droplet and the liquid portion forming a main droplet changes with the amplitude of the harmonic.

FIG. 10 is a diagram for explaining an example of an amplitude determination process.

FIG. 11 is a diagram for explaining an example of an amplitude determination process.

FIG. 12A is an example of a flowchart of a feedback control process.

FIG. 12B is a diagram for explaining a change in the BOP and a change in ΔBOP.

FIG. 13 is a diagram for explaining an example of adjustment of the BOP and/or ΔBOP.

FIG. 14 is a diagram for explaining the state of the charge in a situation where bonding between the liquid portion forming a main droplet and the liquid portion forming a satellite droplet is insufficient.

FIG. 15 is a diagram for explaining the state of the charge in a situation where bonding between the liquid portion forming a main droplet and the liquid portion forming a satellite droplet is sufficient.

FIG. 16 is a diagram for explaining the state of the charge in a situation where bonding between the liquid portion forming a main droplet and the liquid portion forming a satellite droplet is insufficient.

FIG. 17 is a diagram illustrating an example configuration of a biological sample analyzer.

### MODE FOR CARRYING OUT THE INVENTION

The following is a description of preferred modes for carrying out the present disclosure. Note that embodiments described below illustrate representative embodiments of the present disclosure, and the scope of the present disclosure is not limited only to these embodiments. Note that the present disclosure will be described in the following order.
1. First embodiment (flow cytometer)
(1) Basic concept of the present disclosure
(2) Example configuration of a flow cytometer
(3) Setting of waveform parameters
(4) Feedback control
(5) Examples
(6) Example configuration of a biological sample analyzer
2. Second embodiment (waveform parameter setting method)
3. Third embodiment (program)

### 1. First Embodiment (Information Processing Apparatus)

### (1) Basic Concept of the Present Disclosure

As described above, a plurality of factors such as a flow rate, an environmental condition, and a particle size, for example, is involved in droplet formation, and droplet formation is easily affected by external disturbance. Therefore, to stabilize droplet formation or stabilize the amount of the electric charge to be applied to droplets, a vibration element may be driven with a voltage signal in which harmonic components are superimposed in addition to the fundamental vibration. The present disclosure provides a technique for appropriately setting waveform parameters of the harmonic to be superimposed.

A flow cytometer according to the present disclosure includes a vibration control system that controls vibration of a vibration element that generates a droplet, and the vibration control system may be designed to drive the vibration element with a signal having a waveform in which a harmonic is superimposed on the waveform of the fundamental frequency. The vibration control system can set the waveform parameters on the basis of a change caused in a satellite droplet by a change in the waveform parameters of the harmonic. Such a vibration control system that sets waveform parameters in this manner enables stable droplet formation.

For example, high-speed droplet formation in which the fundamental frequency is 100 kHz or higher is more likely to be affected by a minute fluctuation in a factor that affects droplet formation as described above, than the case of droplet formation at normal speed. According to the present disclosure, it is possible to appropriately set the waveform parameters of the harmonic to be adopted in such high-speed droplet formation. That is, in a case where high-speed droplet formation is performed, the flow cytometer according to the present disclosure may set the waveform parameters of the harmonic according to the present disclosure.

In one embodiment, the vibration control system may set the phase of the harmonic, the amplitude of the harmonic, or both the phase and the amplitude of the harmonic, on the basis of a satellite droplet in an image of a generated droplet. In a particularly preferred embodiment, the vibration control system may be designed to set the phase of the harmonic on the basis of a satellite droplet in an image of a generated droplet, and then set the amplitude of the harmonic on the basis of a satellite droplet in an image of a droplet generated in a case where a harmonic having the set phase is adopted. Performing the phase setting process followed by the amplitude setting process in this manner is particularly preferable for setting appropriate waveform parameters.

Further, in the phase setting process, phase setting may be performed on the basis of the state of a Fast satellite, the state of a Slow satellite, or the states of both satellites. The state of a Fast satellite is useful for setting an appropriate phase. Furthermore, in some cases, there is room for improvement in the waveform parameters that have been set only on the basis of the state of a Fast satellite, depending on the structure of the device and the flow rate conditions, for example. In such a case, the vibration control system can perform phase setting on the basis of the state of a Slow satellite. Thus, more appropriate waveform parameters can be set.

That is, according to the present disclosure, when harmonic components are superimposed in addition to the fundamental vibration, droplet images in which phases of the harmonic are swept are analyzed. As a result, the phase of the Fast satellite image or the Slow satellite image in which recovery of the satellite droplet into the main droplet is the quickest is identified. Next, in a state where the identified phase is adopted, images obtained by sweeping amplitudes of the harmonic are analyzed. As a result, the condition for generating a droplet having a preferred state of bonding between the liquid portion forming a satellite droplet and the liquid portion forming a main droplet is identified. In this manner, a phase and an amplitude that are appropriate as waveform parameters for stably generating droplets can be identified.

In one embodiment, the phase of the Fast satellite image in which recovery of a satellite droplet to the main droplet is the quickest is first identified, and images obtained by sweeping amplitudes of the harmonic in a state where the identified phase is adopted may be analyzed. Further, in a case where a preferred droplet generation condition is not identified in the analysis, the phase may be changed to the phase of the Slow satellite in which recovery of the satellite droplet into the main droplet is the quickest. Further, in a state where the identified phase is adopted, images obtained by sweeping amplitudes of the harmonic may be analyzed. Thus, it is possible to cope with a case where an appropriate amplitude is not identified in the phase of a Fast satellite image.

Furthermore, the sorting process is performed for a long time in some cases as described above. In one embodiment, the flow cytometer may be designed to perform a feedback control process for waveform parameters. The feedback control process may be performed so as to adjust (particularly maintain) the position (Brake Off Point, BOP) at which a droplet is separated from the liquid column and/or a distance (ΔBOP) between the position and the separated droplet. This makes it possible to maintain the position at which a droplet separates from the liquid column and/or an inter-droplet distance.

Also, the feedback control process may be performed so as to maintain the liquid width of the liquid portion forming a satellite droplet and the liquid portion forming a main droplet. This enables stable droplet charging.

In a case where the sorting process is performed for a long time, a factor that affects droplet formation might change. However, by the feedback control, it is possible to appropriately cope with such a change.

Further, in the feedback control process, the amplitude of a combined wave obtained by superimposing a harmonic on the fundamental wave may be adjusted. By the adjustment, the BOP (Break Off Point) and/or ΔBOP can be adjusted. The feedback control process may be performed when amplitudes of the harmonic are swept to acquire droplet images, for example. Also, the feedback control process may be performed when phases of the harmonic are swept to acquire droplet images. As the BOP and/or ΔBOP is adjusted by the feedback control process, droplet images can be appropriately compared.

Furthermore, in the feedback control process, the amplitude of the harmonic may be adjusted. Thus, the liquid width of the liquid portion forming the satellite droplet and the liquid portion forming the main droplet can be maintained.

According to the present disclosure, the effect of the two waveform parameters (phase and amplitude) related to the harmonic on the droplet shape becomes clear. Thus, the waveform parameters of the harmonic can be adjusted according to predetermined procedures. Also, it is possible to easily identify the droplet conditions under which the timings to charge a satellite droplet and a main droplet are the same. Accordingly, it is possible to cope with external disturbance such as a temperature change and a pressure fluctuation, for example, and reduce the amount of change in the side stream.

Further, as described above, the phase is changed from the phase of the Fast satellite image in which recovery of the satellite droplet into the main droplets is the quickest to the phase of the Slow satellite in which recovery of the satellite droplet into the main droplet is the quickest. As a result, even in a case where a preferred condition is not identified with a Fast satellite due to the environment or the device-specific condition, it is possible to switch to a Slow satellite and identify a droplet with which the electric charge is stabilized. Thus, the probability of an occurrence of an event in which the droplet generation conditions cannot be adjusted is lowered. Note that the order of the phases to be adopted may be reversed. In other words, the phase may be changed from the phase of the Slow satellite image in which recovery of the satellite droplet into the main droplet is the quickest to the phase of the Fast satellite in which recovery of the satellite droplet into the main droplet is the quickest.

Also, the BOP and/or ΔBOP can be maintained by the feedback control process according to the present disclosure. As a result, the image acquisition timings become the same when the positional relationship or the state of bonding between a satellite droplet and a main droplet is analyzed on the basis of droplet images. Thus, quantitative comparison of numerical values becomes easier. Furthermore, by the feedback control process according to the present disclosure, long-time cell sorting can be stably performed.

### (2) Example Configuration of a Flow Cytometer

A flow cytometer according to the present disclosure includes a vibration control system that controls vibration of a vibration element that generates droplets. The vibration control system may be designed to drive the vibration element with a signal having a waveform in which a harmonic is superimposed on the waveform of the fundamental frequency.

### (2-1) Vibration Control System

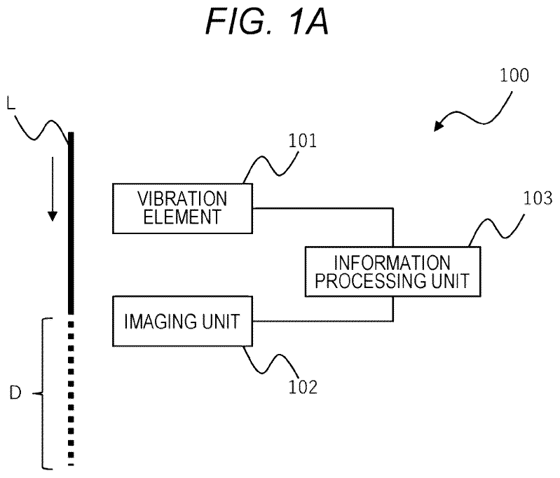

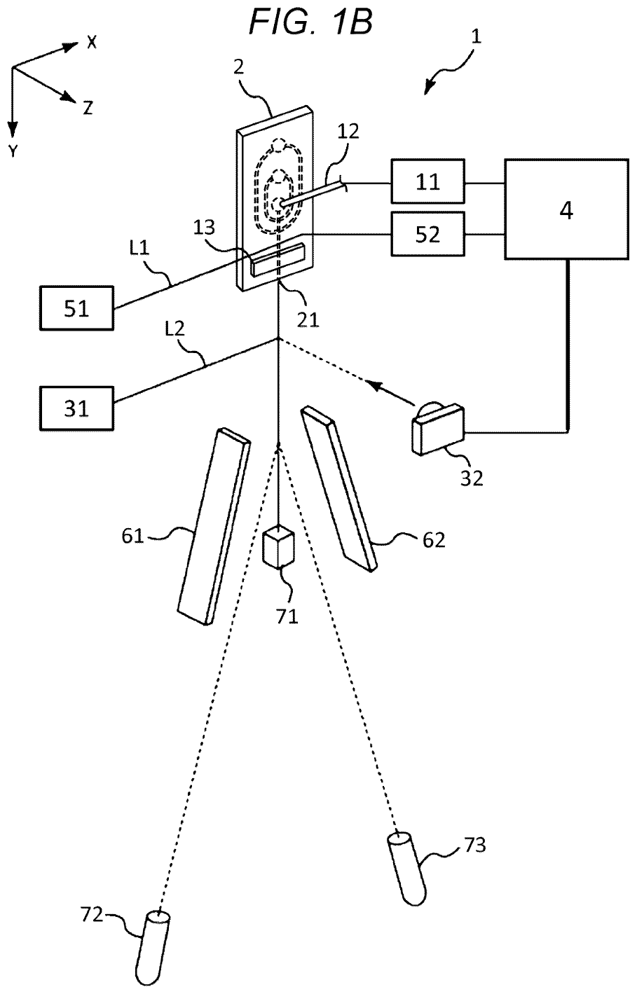

An example configuration of the vibration control system is now described with reference to FIG. 1A. As illustrated in the drawing, a vibration control system 100 according to the present disclosure may include a vibration element 101 that generates droplets, an imaging unit 102 that images a state of droplet formation by the vibration element, and an information processing unit 103 that controls the vibration element.

The vibration element 101 applies vibration to a liquid discharged from a microchip, a flow cell, or the like attached to the flow cytometer. As a result, droplets are formed from the discharged liquid column. In the drawing, the liquid column is indicated by a line denoted by L, and its traveling direction is indicated by an arrow. Furthermore, droplets formed from the liquid column are indicated by a dotted line denoted by D.

The imaging unit 102 is designed to image a state in which a droplet is formed from the liquid column. The imaging unit may include a camera (also referred to as a droplet camera) designed to enlarge and capture an image of a formed droplet, for example, and a strobe light source for instantaneous image capturing.

The information processing unit 103 controls vibration of the vibration element 101. The information processing unit can control the voltage signal to be applied to the vibration element, to control vibration. To generate the voltage signal, the information processing unit may include a vibration element drive signal generation unit, for example. The vibration element drive signal generation unit may be connected to the information processing unit. Also, the information processing unit controls imaging that is performed by the imaging unit 102. Furthermore, the information processing unit performs a waveform parameter setting process according to the present disclosure, on the basis of a droplet image acquired by the imaging. An example of the waveform parameter setting process will be described later in detail. The vibration control system can stabilize droplet formation by the waveform parameter setting process. That is, the vibration control system is also called a droplet stabilization control system.

The information processing unit may perform control to synchronize the vibration element, the vibration element drive signal generation unit, the camera, and the strobe light source. Also, the information processing unit may be designed to adjust the phase of a signal generated by the signal generation unit.

The information processing unit may be designed to drive the vibration element with a signal having a waveform in which a harmonic is superimposed on the waveform of the fundamental frequency. The information processing unit may be designed to be capable of adjusting waveform parameters of the fundamental frequency and waveform parameters of the harmonic. The waveform parameters may be frequency, amplitude, and phase, for example. The information processing unit can control or adjust the waveform parameters of the fundamental frequency and the waveform parameters of the harmonic independently of each other. Furthermore, the information processing unit can control the frequency, the amplitude, and the phase of each waveform parameter independently of each other.

An example configuration of a flow cytometer having the vibration control system is now described with reference to FIG. 1B. The drawing is a schematic diagram of an example configuration of a flow cytometer 1 according to the present disclosure. The flow cytometer 1 includes a chip 2 (also called a microchip) in which a flow channel is formed so as to eject a fluid stream, a vibration element 13, a charging unit 11, an imaging unit 3 (including a strobe 31 and a droplet camera 32), and an information processing unit 4 (that corresponds to the information processing unit 103, and is also called a control unit).

The flow cytometer 1 may further include a light irradiation unit 51, a detection unit 52, and deflection plates 61 and 62. The flow cytometer 1 may further include recovery vessels 71 to 73 that may be attached thereto in a replaceable manner. The information processing unit 4 may include an analysis unit, a storage unit, a display unit, an input unit, and the like, for example. In the description below, these components will be described.

### (2-2) Chip 2

The chip 2 may have a flow channel designed to form a fluid (particularly, a laminar flow) including a sample flow containing microparticles and a sheath flow flowing so as to enclose the sample flow. The chip 2 may be replaceable. That is, the chip 2 can be designed to be detachable from the flow cytometer 1. Also, the flow cytometer 1 may have a flow cell or a cuvette attached thereto, instead of the chip 2, and the flow cell or the cuvette may have a flow channel designed to form the fluid. The chip, the cuvette, or the flow cell may be formed with a plastic material or a glass material. The flow channel may be formed in a substrate formed with such a material.

The chip 2 has an orifice 21 through which the fluid is ejected. The fluid stream ejected from the orifice 21 is turned into droplets by vibration applied by the vibration element 13. For example, the vibration element 13 applies the vibration to the orifice 21, and the droplet formation is performed.

The vibration element 13 may be a piezoelectric element, for example, but is not limited to this. The vibration element 13 applies vibration to the fluid, to form droplets in the fluid. The vibration element 13 may be positioned so as to be in contact with the liquid in the flow channel. The fluid flow rate and the orifice diameter may be adjusted as appropriate.

The droplets formed by the flow cytometer may include main droplets and satellite droplets. The main droplets are droplets formed by surface tension from a rod-shaped liquid column of the fluid discharged from the orifice, and the main droplets contain particles. The satellite droplets are small droplets generated in conjunction with the formation of the main droplets. Since the satellite droplets can cause fluctuations in the amount of charge to be applied to the main droplets, control on the satellite droplets is required. Control on the satellite droplets is particularly important for the flow cytometer that requires highly-accurate droplet deflection position control.

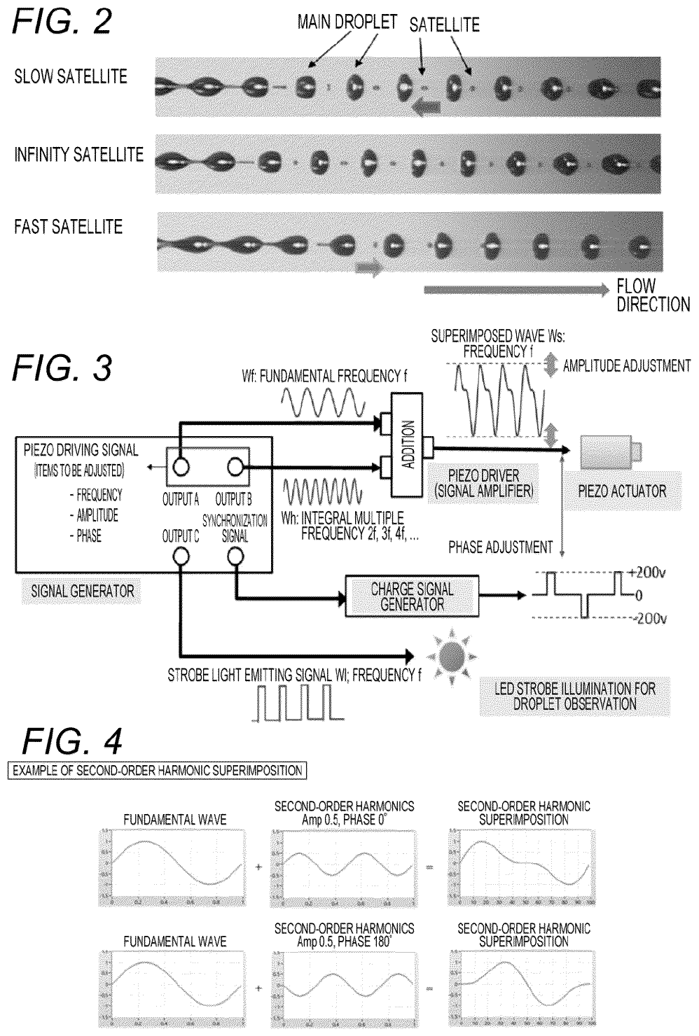

The satellite droplets can be divided into the following four types: Fast satellites (also referred to as Forward satellites), Slow satellites (also referred to as Back satellites), Infinity, and Non-satellites. These satellite droplets are now described with reference to FIG. 2.

As illustrated in the drawing, a Fast satellite is a satellite droplet that is formed so that the upstream-side end (on the upstream side in the flow direction) of the liquid portion forming the satellite droplet is separated from the main droplet (referred to as the upstream-side main droplet), and the downstream-side end (on the downstream side in the flow direction) of the liquid portion forming the satellite droplet is then separated from the main droplet (also referred to as the downstream-side main droplet) flowing one droplet ahead of the main droplet. The Fast satellite gradually approaches the downstream-side main droplet, and is absorbed by the downstream-side main droplet.

Note that the upstream-side end may mean an end closer to the orifice. Of the two ends of the satellite droplet, the downstream-side end may mean the end farther from the orifice.

As illustrated in the drawing, a Slow satellite is a satellite droplet that is formed so that the downstream-side end of the liquid portion forming the satellite droplet is separated from the downstream-side main droplet, and the upstream-side end of the liquid portion forming the satellite droplet is then separated from the upstream-side main droplet. The Slow satellite gradually approaches the upstream-side main droplet, and is absorbed by the upstream-side main droplet.

Infinity involves a satellite droplet in which the lower end and the upper end of the satellite droplet are almost simultaneously formed away from the two main droplets sandwiching the satellite droplet, and the satellite droplet travels without being absorbed by either of the upstream-side main droplet and the downstream-side main droplet. That is, Infinity may mean a case where separation occurs while there is almost no difference in drop velocity between the satellite droplets and the main droplets.

Non-satellites mean a liquid in a case where any satellite droplet separated from two main droplets is not formed but is absorbed by either of the main droplets. For example, a case where the upstream-side end of the liquid portion forming a satellite droplet is separated from one main droplet, but the downstream-side end is absorbed by the other main droplet before being separated from the other main droplet is a Non satellite.

### (2-3) Charging Unit 11

The charging unit 11 is designed to charge droplets containing the microparticles with a positive or negative electric charge. That is, the charging unit 11 applies an electric charge to droplets discharged from the orifice 21. For example, as illustrated in FIG. 1B, the charging unit 11 may be disposed to charge a fluid on the upstream side of the point of imaging by the imaging unit 3 described later. Charging of droplets can be performed with an electrode 12 electrically connected to the charging unit 11. Note that the electrode 12 may be inserted at some location so as to be in electrical contact with the sample liquid or the sheath liquid being supplied through the flow channel.

For example, the flow cytometer 1 may be designed so that the charging unit 11 charges droplets containing the microparticles, after a drop delay time has passed since the microparticles contained in the sample liquid were detected by a detection unit 5 described later.

### (2-4) Imaging Unit 3

The imaging unit 3 may be designed to image a state in which a droplet is formed from a discharged liquid column. That is, as illustrated in FIG. 2 and other drawings, the imaging unit captures an image of a liquid column and the region covering a droplet generated from the liquid column. The imaging unit 3 may be designed to capture a droplet image (photograph) at an any time or in any phase. The imaging unit 3 includes the strobe 31 and the droplet camera 32, for example.

The droplet camera 32 may be disposed so as to be able to image a state in which a droplet is formed from a fluid stream ejected from an orifice. That is, the droplet camera 32 may be designed to capture an image of a liquid column and the region covering a droplet. The droplet camera 32 may be a CCD camera or a CMOS sensor, for example. The droplet camera 32 is disposed so as to perform imaging on the downstream side of the position of light irradiation by the detection unit 5 described later. Also, the droplet camera 32 may be a camera that can perform focus adjustment so as to adjust the point of focus for imaging onto a droplet. As for the light source that irradiates the subject (droplet) in imaging by the droplet camera 32 with light, the strobe 31 described later may be used, for example.

The strobe 31 may be an LED for imaging a droplet. Also, the strobe 31 may include a laser L2 (a red laser light source, for example) for imaging microparticles. The light source of the strobe 31 may be switched in accordance with the purpose of imaging.

In a case where an LED is used as the strobe 31, the LED may emit light only for a very short time of one cycle of a droplet frequency (Droplet CLK). The droplet frequency corresponds to the fundamental frequency described later. The light emission may be performed in each cycle of the droplet frequency, and thus, a certain moment of droplet formation can be cut out and acquired as an image. While imaging by the droplet camera 32 is performed about 30 times per second, for example, the droplet frequency may be about 10 kHz to 100 kHz.

### (2-5) Deflection Plates

Reference signs 61 and 62 in FIG. 1B indicate a pair of deflection plates disposed to face each other, with a droplet ejected from the orifice 21 and imaged by the imaging unit 3 being interposed in between. The deflection plates 61 and 62 include electrodes that control the moving direction of the droplet discharged from the orifice 21 with an electric acting force that acts with the electric charge applied to the droplet. Furthermore, the deflection plates 61 and 62 control the trajectory of the droplet generated from the orifice 21 with an electric acting force that acts with the electric charge applied to the droplet. In FIG. 1B, the facing direction of the deflection plates 61 and 62 is indicated by the X-axis direction.

### (2-6) Vibration Element Drive Signal Generation Unit

An example configuration of the vibration element drive signal generation unit included in the flow cytometer 1 is now described with reference to FIG. 3. The drawing is a conceptual diagram for explaining driving of the vibration element 13 for generating droplets. As described above, the vibration control system drives the vibration element with a combined waveform obtained by superimposing a harmonic of a frequency of an integer multiple on the waveform (sine waves, for example) of the fundamental frequency. By superimposing high-frequency waves, the droplet shape can be manipulated with high accuracy. The signal generation unit illustrated in the drawing includes a signal generator. The signal generator has output ends A and B (shown as an “output A” and an “output B”, respectively) that output signals for driving the vibration element (a piezo actuator). The signal generator outputs a signal having the waveform of the fundamental frequency from the output end A to a vibration element drive unit (a piezo driver), and outputs a signal having the waveform of the harmonic from the output end B to the vibration element drive unit. The vibration element drive unit outputs a signal having a combined waveform in which these two signals are superimposed on each other to the vibration element. Note that the configuration of the signal generation unit is not limited to that illustrated in the drawing, and may be changed as appropriate by a person skilled in the art.

The signal generator may be designed to be able to adjust the waveform parameters (the frequency, the amplitude, and the phase of the signal of the fundamental frequency and the waveform parameters (the frequency, the amplitude, and the phase) of the signal of the harmonic, independently of each other.

The signal generator may be designed to output a signal to a charge signal generator shown in the drawing. The charge signal generator generates a signal for charging by the charging unit described above. Further, the signal generator may be designed to output a signal to a strobe (strobe illumination for droplet observation). The frequency of the signal to the strobe may be the same as the fundamental frequency. With such a configuration, the signal generator can synchronize the vibration by the vibration element with the charging by the charging unit and/or the light irradiation by the strobe. In this manner, the waveform of the signal for driving the vibration element is synchronized with the imaging unit for droplet observation and/or the charging unit. By such synchronization, the target droplets can be sorted.

As illustrated in FIG. 4, the combined waveform of the fundamental wave and the harmonic may be characterized by the two parameters of the amplitude and the phase of the harmonic to be superimposed.

As illustrated in the upper portion of the drawing, a signal having a superimposed waveform illustrated at the upper right of the drawing is generated by superimposing a harmonic (a second-order harmonic, Amp: 0.5, phase: 0°) on the fundamental wave. Also, as illustrated in the lower portion of the drawing, a signal having a superimposed waveform illustrated at the upper right of the drawing is generated by superimposing a harmonic (a second-order harmonic, Amp: 0.5, phase: 180°) on the fundamental wave. By changing the phase of the harmonic in this manner, it is possible to adjust the waveform of the superimposed wave.

Likewise, by changing the amplitude of the harmonic, it is also possible to adjust the waveform of the superimposed wave.

### (2-7) Light Irradiation Unit 51 and Detection Unit 52

The microparticles flowing in the flow channel are irradiated with laser light L1 emitted from the light source of the light irradiation unit 51. The detection unit 5 detects the measurement target light generated by the irradiation. On the basis of the detected light, the information processing unit 4 analyzes the microparticles in the fluid flowing in the flow channel. As for example configurations of the light irradiation unit 51 and the detection unit 52, the description regarding the light irradiation unit and the detection unit included in a biological sample analyzer 6100 described later applies, and therefore, it is preferable to refer to that.

### (2-8) Recovery Vessels 71 to 73

In the flow cytometer 1, droplets are received in any one recovery vessel of a plurality of recovery vessels 71 to 73 arranged in a line in the facing direction (X-axis direction) of the deflection plates 61 and 62. The recovery vessels 71 to 73 may be general-purpose plastic tubes or glass tubes for experiment. The number of recovery vessels 71 to 73 is not limited to any particular number, but a case where three recovery vessels are installed is illustrated in the drawing. A droplet generated from the orifice 21 is guided to one of the three recovery vessels 71 to 73 and is recovered, depending on the presence or absence of an electrical acting force between the deflection plates 61 and 62, and the magnitude thereof.

The recovery vessels 71 to 73 may be replaceably installed in a container for recovery vessels (not illustrated). The container for recovery vessels is disposed on a Z-axis stage (not illustrated) designed to be movable in a direction (Z-axis direction) orthogonal to the direction of droplet discharge (Y-axis direction) from the orifice 21 and the facing direction (X-axis direction) of the deflection plates 61 and 62, for example.

### (2-9) Information Processing Unit 4

The information processing unit 4 corresponds to the information processing unit 103 described above with reference to FIG. 1A. As for an example configuration of the information processing unit 4, the description regarding the information processing unit included in the biological sample analyzer 6100 described later applies, and therefore, it is preferable to refer to that.

The information processing unit 4 may include an analysis unit, a storage unit, a display unit, an input unit, and the like, for example. The analysis unit can analyze light detected by the detection unit 52. On the basis of the analysis result, the information processing unit 4 can determine whether to sort particles. The storage unit can store a value and the like detected by the detection unit 52.

The display unit can display data related to waveform parameter setting, for example. The data may include the waveform data (the waveform and the waveform parameters, for example) of the fundamental wave, the waveform data of the harmonic, the waveform data of a superimposed wave, and the like, for example. Also, the display unit can display a droplet image captured by the imaging unit. The display unit may include a display device, for example, and the configuration thereof may be selected as appropriate.

The input unit receives a data input from a user. The input unit may include a touch panel, a mouse, or a keyboard, for example.

Furthermore, the information processing unit 4 may include a program for causing a flow cytometer (particularly, the vibration control system) to perform a waveform parameter setting process according to the present disclosure. The program can be stored in the storage unit, for example.

### (3) Setting of Waveform Parameters

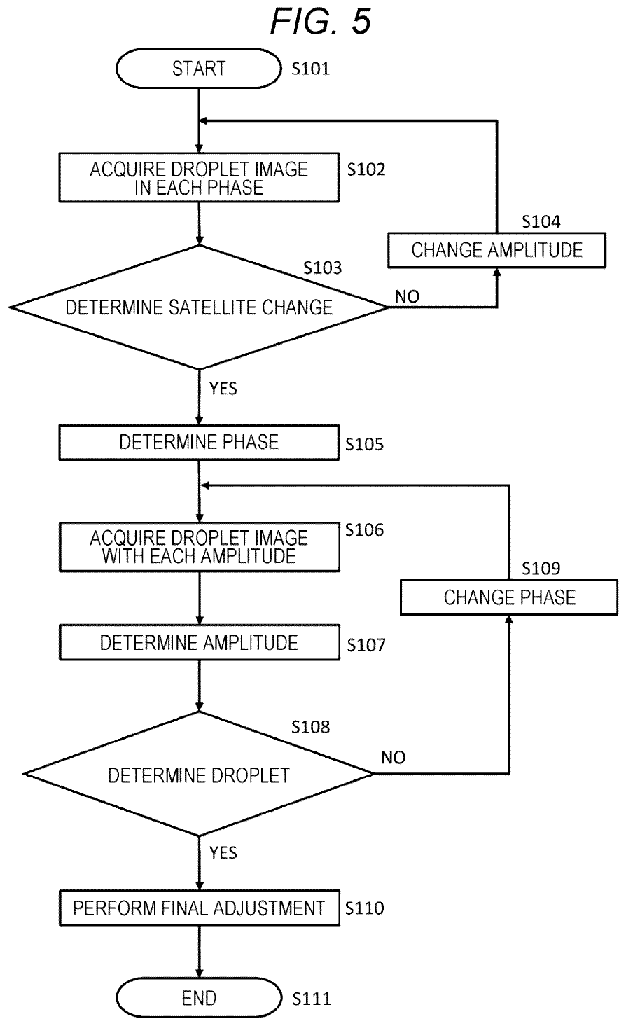

In the description below, an example of the waveform parameter setting process to be performed by the flow cytometer 1 according to the present disclosure will be described with reference to FIG. 5. The drawing is an example of a flowchart of the process.

### (Step S101)

In step S101, the flow cytometer 1 (particularly, the vibration control system 100) starts the waveform parameter setting process. As the process starts, a liquid is discharged from an orifice of the chip. The liquid forms a liquid column.

### (Step S102)

In step S102, the vibration control system drives the vibration element with a signal having a superimposed waveform in which a harmonic is superimposed on the waveform of the fundamental frequency (also referred to as the fundamental wave). As a result, vibration is applied to the liquid column discharged from the orifice, and droplets are formed.

In step S102, the droplet camera captures an image of a state in which a droplet is formed. To capture an image of a state in which a droplet is formed, the droplet camera captures an image of a predetermined region covering the range in which a droplet is formed from the liquid column discharged from the orifice.

In step S102, the waveform parameters (the frequency, the amplitude, and the phase) of the fundamental wave may be fixed. Meanwhile, regarding the waveform parameters of the harmonic, the frequency and the amplitude are fixed, but the phase is changed. In each of the changed phases, the droplet camera captures an image of a state in which a droplet is formed. In this manner, an image (also referred to as a droplet image) of the droplet forming state in each phase is acquired.

For example, the phase difference between the harmonic and the fundamental wave may be changed so as to sweep all one period of the phase of the harmonic, for example, and may be changed at predetermined intervals in the range of 0° to 360°, for example.

In the same step, in a case where the amplitude of the fundamental wave is 1, the amplitude of the harmonic may be fixed to any value from 0.01 to 0.4, or particularly, to any value from 0.1 to 0.3, for example. As an example, the amplitude of the harmonic may be about 0.2 in a case where the amplitude of the fundamental wave is 1.

In the same step, the frequency of the fundamental wave may be 1 khz or higher, for example, or preferably, 10 KHz or higher, 30 KHz or higher, or 50 kHz or higher. Furthermore, the frequency of the fundamental wave may be 500 kHz or lower, for example, or preferably, 200 kHz or lower, 180 kHz or lower, or 150 kHz or lower.

In the same step, the frequency of the harmonic may be at least twice the frequency of the fundamental wave, for example. Furthermore, the frequency of the harmonic may be equal to or lower than five times the frequency of the fundamental wave, for example, or preferably, be equal to or lower than four times the frequency of the fundamental wave.

As described above, in the same step, the vibration control system (particularly, the information processing unit) drives the vibration element with each of signals having various superimposed waveforms that vary only in the phase of the harmonic, and causes the droplet camera to capture an image of a state of a droplet formed with each signal. In this manner, the vibration control system acquires an image indicating the state of droplet formation with each signal.

Preferably, in a case where a droplet image is acquired while the waveform parameters of the harmonic are changed, the vibration control system (particularly, the information processing unit) may perform the superimposed waveform change in step S102, so as to maintain the position of the BOP and/or maintain the distance (ΔBOP) between the BOP and the separated droplet. The maintenance of the BOP and/or ΔBOP may be performed as described later in “(4) Feedback control”.

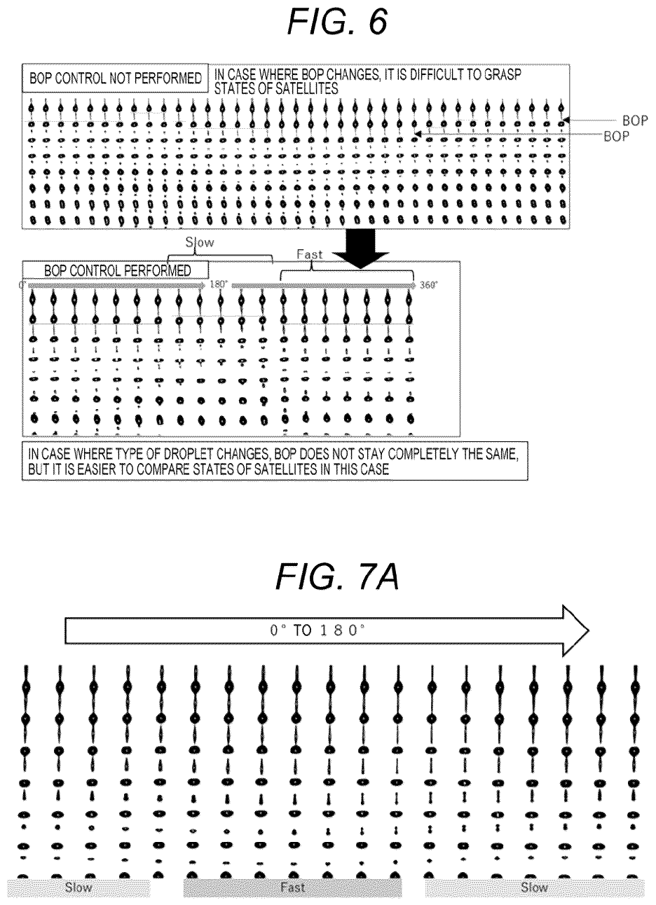

The maintenance of the BOP and/or ΔBOP is now described with reference to FIG. 6.

As illustrated in the upper portion of the drawing, when the vibration element is driven with a signal having a superimposed waveform in which the waveform parameters of the fundamental wave are fixed but the waveform parameters of the harmonic are changed in a various manner, and a droplet image in the case with each signal is acquired, the position (Brake Off Point: BOP) at which a droplet is separated from the liquid column greatly changes. Since the timing for a droplet to be separated is not fixed, droplet images acquired in this manner are not suitable for quantitatively determining the relationship between satellite droplets and the main droplets.

As illustrated in the lower portion of the drawing, in a series of droplet images captured so that the BOP and/or ΔBOP is maintained, the timing of separation of a droplet from the liquid column is fixed, and thus, satellite determination can be appropriately performed. For example, the types of satellite droplets can be appropriately classified. It is also possible to quantitatively analyze a change in timing at which a satellite droplet is recovered into the main droplet.

Note that these maintenance operations may not be performed between a case with a Fast satellite and a case with a Slow satellite. Also, in step S102, the BOP position and ΔBOP do not have to be maintained so as to be completely the same, and are only required to be maintained to such an extent that the determination process described later can be appropriately performed.

### (Step S103)

In step S103, the vibration control system (particularly, the information processing unit) determines whether the droplet image has changed with the change in phase, on the basis of the images acquired in step S102.

For example, with the harmonic amplitude set in step S102, the droplet image may not change even if the phase is changed in some cases. In such cases, even if the processes in and after step S105 are performed, a situation in which appropriate waveform parameter setting cannot be performed might occur.

In a case where, in the above determination, the droplet image is determined not to have changed, the vibration control system advances the process to step S102.

In a case where, in the above determination, the droplet image is determined not to have changed, the vibration control system advances the process to step S104.

Therefore, it is determined in step S103 whether there is a change in the droplet image. If there is not a change, the amplitude is changed in step S104, and step S102 is then carried out again. Thus, it is possible to prevent a situation in which appropriate waveform parameters are not set due to execution of the processes in and after step S105.

### (Step S104)

In step S104, the vibration control system (particularly, the information processing unit) changes the amplitude of the harmonic adopted in step S102, and reconfigures the superimposed waveform to be adopted in step S102. In step S104, the waveform parameters of the fundamental wave may not be changed. Also, in step S104, the other waveform parameters (particularly, the frequency) of the harmonic may not be changed.

For example, in step S104, the vibration control system can adopt, as the amplitude of the harmonic, an amplitude value obtained by adding a predetermined value to (or subtracting the predetermined value from) the amplitude of the harmonic adopted in step S102. The predetermined value may be set as appropriate by a person skilled in the art in accordance with factors such as the device configuration or the droplet to be formed, for example.

### (Step S105)

In step S105, the vibration control system (particularly, the information processing unit) performs a phase determination process to determine the phase of the harmonic, on the basis of the droplet images acquired in step S102.

For example, the vibration control system performs the process on the basis of the droplet images acquired in step S102 so that the timing at which a satellite droplet is absorbed by a main droplet is further advanced. The timing being earlier is preferable for electrical control on the droplet traveling direction, and contributes to appropriate execution of a sorting process. Also, the timing being earlier means that a situation with only small satellites that are easily affected by external disturbance is resolved early, and thus, the side stream is stabilized. Further, it is also less likely to be affected by a repulsive force or an attractive force caused by an electric charge between droplets.

In one embodiment, the phase determination process may include a classification process of classifying types of satellite droplets in each droplet image on the basis of the droplet images acquired in step S102, and a phase identification process of identifying an optimum phase on the basis of a classification result.

The classification process may include a first classification process of classifying each droplet in a droplet group in a droplet image as a main droplet or a satellite droplet, and a second classification process of classifying the types of the droplets classified as satellite droplets. In the second classification process, the type of a droplet may be classified as either of two types of Fast satellite and Slow satellite, for example, or may be classified as any one of three types or four types of Infinity and/or Non satellite in addition to these two types as necessary.

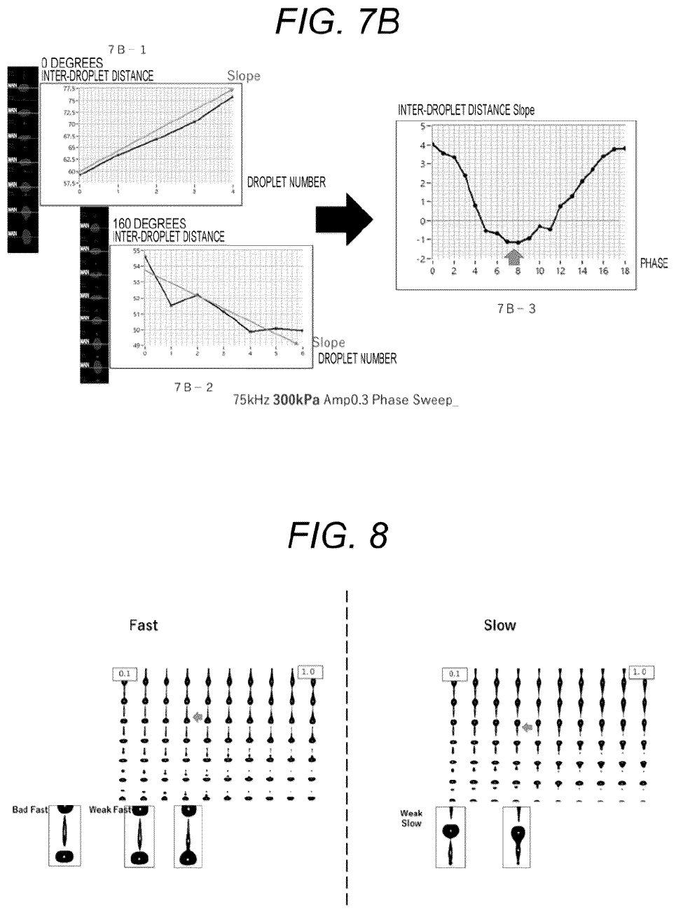

The first classification process may be performed on the basis of the size of each droplet in the droplet image, for example. As illustrated in FIG. 7A, a main droplet and a satellite droplet have different sizes, and therefore, the classification can be performed through image processing. For example, the classification may be performed on the basis of the size (such as the width, for example) or the area of a droplet.

The second classification process may be performed on the basis of a change in the distance between a main droplet and a satellite droplet, for example. As illustrated in the drawing, a plurality of main droplets and a plurality of satellite droplets are present in a droplet image in a certain phase.

In a case where a satellite droplet is absorbed by the main droplet (Fast in FIG. 7A) in front of the satellite droplet (or the main droplet flowing one droplet before the satellite droplet), the satellite droplet is a Fast satellite.

In a case where a satellite droplet is absorbed by the main droplet (Slow in FIG. 7A) behind the satellite droplet (or the main droplet flowing one droplet behind the satellite droplet), the satellite droplet is a Slow satellite.

For example, as for a Fast satellite, the distance between the satellite droplet and the main droplet in front thereof becomes gradually shorter. Therefore, as for a Fast satellite, the slope representing the change in the distance is a negative value, as illustrated in FIG. 7B-2 (phase 160°), for example. The slope representing the change in the distance may be the slope of a plot of the distance with respect to each position in the droplet flow direction or the droplet number in the droplet image (such as the droplet number increasing from the upstream toward downstream, for example).

As for a Slow satellite, on the other hand, the distance between the satellite droplet and the main droplet in front thereof becomes gradually longer. Therefore, as for a Slow satellite, as illustrated in FIG. 7B-1 (phase 0°), the slope representing the change in the distance (or the slope of the plot of the distance with respect to the position in the droplet flow direction, for example) has a positive value.

Accordingly, in each droplet image, the type of a satellite droplet can be identified on the basis of the change in the distance (or the slope representing the change, for example).

For each of the droplet images acquired in step S102, for example, the vibration control system classifies the type of the satellite droplets, on the basis of the distance between a satellite droplet and the main droplet in front thereof. In particular, for each of the droplet images acquired in step S102, the vibration control system determines whether a satellite droplet is a Fast satellite or a Slow satellite (alternatively, one of three or four kinds of Infinity and/or Non satellite in addition to Fast and Slow is used as necessary), on the basis of the distance between the satellite droplet and the main droplet in front thereof. In this manner, the type of the satellites in each droplet image is identified.

In one embodiment, in the phase identification process, the vibration control system selects the droplet image in which a satellite droplet is absorbed by a main droplet at the earliest timing from among the droplet images showing Fast satellites, for example, and identifies the phase in which the selected droplet image has been acquired.

In this embodiment, the vibration control system may select the droplet image in which a satellite droplet is absorbed by a main droplet at the earliest timing from among the droplet images showing Slow satellites, and identify the phase in which the selected droplet image has been acquired.

That is, the vibration control system may select the droplet image in which a satellite droplet is absorbed by a main droplet at the earliest timing from among the droplet images showing Fast satellites, or may select the droplet image in which a satellite droplet is absorbed by a main droplet at the earliest timing from among both the droplet images showing Fast satellites and the droplet images showing Slow satellites, and identify the phase in which the selected droplet image has been acquired.

In another embodiment, in the phase identification process, the vibration control system selects the droplet image in which a satellite droplet is absorbed by a main droplet at the earliest timing from among the droplet images showing Slow satellites, for example, and identifies the phase in which the selected droplet image has been acquired.

In this manner, the vibration control system identifies the phase in which a satellite droplet is absorbed by a main droplet at the earliest timing in a case with Fast satellites or a case with Slow satellites or a case with both types of satellites.

In a preferred embodiment, the vibration control system identifies the phase in which a satellite droplet is absorbed by a main droplet at the earliest timing, both in a case with Fast satellites and a case with Slow satellites. As a result, in a case where an appropriate harmonic amplitude cannot be identified in the phase of one type of satellite in a process to be described later, the phase of the other type of satellite can be adopted to set an appropriate harmonic amplitude.

For example, for the phase identification process, the change in the distance (the slope representing the change, for example) described above can be used. That is, on the basis of the change in the distance (the slope representing the change, for example) described above, the vibration control system identifies the phase in which a satellite droplet is absorbed by a main droplet at the earliest timing in a case with Fast satellites or a case with Slow satellites or a case with both types of satellites.

An example of the identification is now described with reference to FIG. 7B.

As described above with reference to FIGS. 7B-1 and 7B-2, the slope value representing the change in the inter-droplet distance is calculated by plotting the inter-droplet distance with respect to droplet numbers in each phase.

Where the slope value representing the change in the distance is plotted with respect to the phases, a plot indicating the fluctuation of the slope value with respect to the phases is obtained as illustrated in FIG. 7B-3. The maximum value and/or the minimum value of the slope value in the plot can be identified. For example, in FIG. 7B-3, there is the minimum value of the slope value at the position indicated by an upward arrow, and the minimum value corresponds to the Fast satellite having the earliest absorption timing. Likewise, the maximum value of the slope value corresponds to the Slow satellite having the earliest absorption timing. Therefore, the vibration control system may identify the phase in the case where the slope value becomes smallest as the phase of the Fast satellite having the earliest absorption timing. Also, the vibration control system may identify the phase in the case where the slope value becomes largest as the phase of the Slow satellite having the earliest absorption timing. The phase identification method may be changed as appropriate, in accordance with a change of the plotting method.

In step S105, the phase specified in this manner may be determined as the phase of the harmonic.

### (Step S106)

In step S106, the vibration control system (particularly, the information processing unit) drives the vibration element with a signal having a superimposed waveform in which a harmonic is superimposed on the waveform of the fundamental frequency (also referred to as the fundamental wave), as in step S102. As a result, vibration is applied to the liquid column discharged from the orifice, and droplets are formed.

In step S106, the droplet camera captures an image of a state in which a droplet is formed, as in step S102. To capture an image of a state in which a droplet is formed, the droplet camera captures an image of a predetermined region covering the range in which a droplet is formed from the liquid column discharged from the orifice.

In step S106, the waveform parameters (the frequency, the amplitude, and the phase) of the fundamental wave may be fixed as in step S102, and the waveform parameters of the fundamental wave may also be the same as those in step S102.

As for the waveform parameters of the harmonic, the frequency may be the same as that in step S102. The phase of the harmonic may be the phase determined in step S105. In step S106, among the waveform parameters of the harmonic, the frequency and the phase are fixed, but the amplitude is changed. With each of the changed amplitudes, the droplet camera captures an image of a droplet (a state in which a droplet is formed).

In the same step, in a case where the amplitude of the fundamental wave is set to 1, the amplitude of the harmonic may be changed within the range of 0.01 to 4, or particularly, within the range of 0.1 to 2, or more particularly, within the range of 0.1 to 1, for example. Such a range is preferred to efficiently identify an optimum amplitude.

As described above, in the same step, the vibration control system drives the vibration element with each of signals having various superimposed waveforms that vary only in the amplitude of the harmonic, and causes the droplet camera to capture an image of a state of a droplet formed with each signal. In this manner, the vibration control system acquires a droplet image with each changed amplitude while changing the amplitude of the harmonic.

An example of droplet images acquired in step S106 is now described with reference to FIG. 8. The droplet images shown in the drawing are captured in an appropriate phase of a Fast satellite (the left side in the drawing, fundamental frequency: 86 kHz, phase: 90°) and a Slow satellite (the right side in the drawing, fundamental frequency: 86 kHz, phase: 170°), with the harmonic amplitude changed from 0.1 to 1.0. Where the portions of the images at the positions where a droplet is separated are enlarged, it can be seen that the state of bonding between the main droplet and the satellite droplet after separation varies with the amplitude of the harmonic (the portions indicated by arrows). To charge the droplets in a stable manner, it is important to separate the liquid portions forming the satellite droplets and the liquid portions forming the main droplets from the liquid column while the liquid portions forming the satellite droplets and the liquid portions forming the main droplets are bonded to each other. Therefore, the amplitude of the harmonic may be set to a value at which the satellite droplet forming portions and the main droplet forming portions are bonded.

### (Step S107)

In step S107, the vibration control system (particularly, the information processing unit) determines a preferable amplitude as a state in which a satellite is recovered to the main droplet, on the basis of the droplet images acquired in step S106. That is, the vibration control system may determine the amplitude of the harmonic, on the basis of the change caused in the satellite droplet image by a change in the amplitude of the harmonic. This is because the amplitude component of the harmonic does not contribute to the classification of satellites, but contributes mainly to the satellite recovery timing. When the amplitude of the harmonic is increased, the satellites are normally recovered by the main droplets in a quick manner. However, if the amplitude is made too large, the distortion of the droplet shape becomes too large. Therefore, it is important to balance the recovery timing and the droplet shape.

In an implementation, in step S107, the vibration control system determines the amplitude of the harmonic so that the liquid portions forming the satellite droplets and the liquid portions forming the main droplets separate from the liquid column while remaining bonded to each other. For example, in step S107, the vibration control system may determine the amplitude of the harmonic on the basis of the width of the liquid. The width of the liquid is the width in a direction perpendicular to the traveling direction (flow direction) of droplets. By referring to the width, it is possible to appropriately determine the state of the bonding.

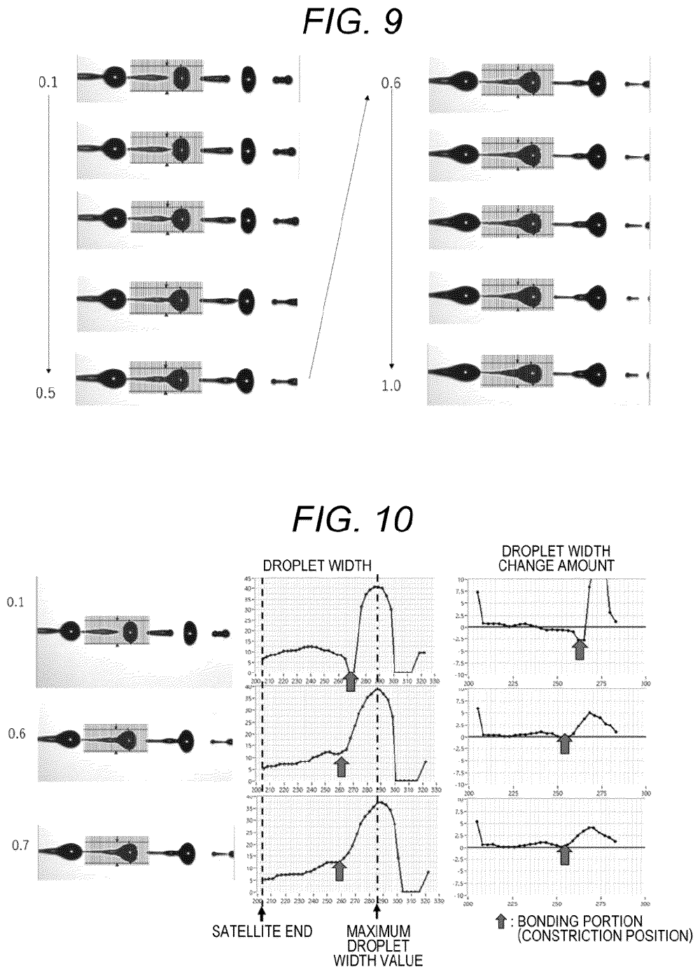

An example of the width-based setting process is now described with reference to FIG. 9. The drawing illustrates that, in a case with a FAST satellite, the state of bonding between the liquid portion forming the satellite droplet and the liquid portion forming the main droplet changes with the amplitude of the harmonic. The drawing illustrates a droplet formation state imaged in a case where the amplitudes of the harmonic are changed from 0.1 to 0.2, . . . , and to 1.0 while the amplitude of the fundamental wave is 1. As illustrated in the drawing, in cases where the amplitude is 0.1 and 0.2, for example, the liquid portion forming a satellite droplet and the liquid portion forming its main droplet are separated from each other, and therefore, these cases are not appropriate. With an amplitude of 0.6, the liquid portion forming a satellite droplet and the liquid portion forming its main droplet are bonded, but the bonding portion is constricted. To stably charge droplets, it is preferable to select an amplitude with which the bonding portion between the liquid portion forming a satellite droplet and the liquid portion forming its main droplet is not constricted (or an amplitude with which the degree of constriction is lower in a case where constriction always occurs). Therefore, in the present disclosure, the vibration control system determines an amplitude that is such that the liquid portion forming a satellite droplet and the liquid portion forming its main droplet are separated from the liquid column while remaining bonded, and such that there is no constriction (or such constriction is smaller) in the bonding portion between the two liquid portions. The vibration control system may refer to the width described above for determining the degree of constriction. The width may be identified by image processing performed on the droplet image. The technique of the image processing may be selected as appropriate by a person skilled in the art.

As described above, the vibration control system may determine the amplitude of the harmonic, on the basis of a change in the state of bonding between the liquid portion forming a satellite droplet and the liquid portion forming its main droplet.

An example of the process of determining the amplitude is now described with reference to FIG. 10. The left column in FIG. 10 shows the droplet images of the states of bonding between the liquid portion forming a satellite droplet and the liquid portion forming its main droplet in the cases with the amplitudes of 0.1, 0.6, and 0.7 among the droplet images shown in FIG. 9. The middle column in FIG. 10 shows plot results obtained by plotting the width of the liquid (droplet width) with respect to the position of the liquid in the traveling direction in each of these cases. The right column in FIG. 10 shows plot results obtained by plotting the amount of change in width of the liquid (the droplet width change amount) with respect to the position of the liquid in the traveling direction.

In the middle column in the drawing, as illustrated regarding the case with the amplitude of 0.7, in a case where the constriction does not occur in the bonding portion, the width of the liquid monotonously increases from the end (satellite end) on the side of the portion forming the satellite droplet to the maximum value (the maximum droplet width value). In a case where constriction appears, on the other hand, the width of the liquid might not increase monotonically but might decrease (the positions indicated by arrows), as illustrated regarding the cases with the amplitudes of 0.1 and 0.6.

Therefore, according to the present disclosure, the vibration control system may select, as the harmonic amplitude, the amplitude in the case where the droplet image in which the width of the liquid monotonously increases from the end on the side of the portion forming the satellite droplet to the maximum value is captured, from among the swept amplitudes.

Further, as illustrated regarding the case with the amplitude of 0.7 in the right column in the drawing, in a case where constriction does not occur at the bonding portion, the amount of change in the width of the liquid does not turn into a negative value between the satellite end and the maximum droplet width value. That is, the amount of change is not negative at the bonding portion indicated by an arrow. In a case where constriction appears, on the other hand, the droplet width change amount turns into a negative value in some cases, as illustrated regarding the cases with the amplitudes of 0.1 and 0.6. That is, the amount of change is negative at the bonding portions indicated by arrows.

Therefore, according to the present disclosure, the vibration control system may select the amplitude in a case where a droplet image in which the amount of change in the width of the liquid is 0 or larger (or more than 0) from the end on the side of the portion forming the satellite droplet to the maximum value is captured.

As described above, the vibration control system may determine the amplitude of the harmonic, on the basis of the width of the bonding portion between the liquid portion forming a satellite droplet and the liquid portion forming its main droplet.

There are cases with no amplitudes with which constriction does not occur in the bonding portion. In such a case, the vibration control system may change the conditions for the amplitude to be selected.

For example, the vibration control system may select the amplitude in a case where the liquid width of the constricted portion is equal to or greater than a predetermined proportion (such as 10% or higher, 20% or higher, or 30% or higher) to the maximum value.

Also, the vibration control system may select the amplitude in a case where the amount of change is a predetermined value or larger (a predetermined negative value or larger, for example).

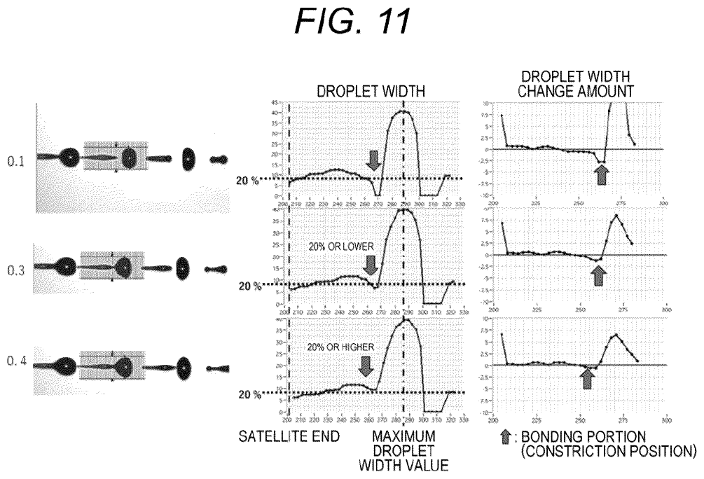

This aspect is now described with reference to FIG. 11. In the drawing, droplet images (the left column), width plots (the center column), and change amount plots (the right column) in cases where the harmonic amplitudes are 0.1, 0.3, and 0.4 with respect to the fundamental wave of 1 are illustrated, as in FIG. 10. In a case with any of the amplitudes, the amount of change might not monotonically increase from the satellite end to the maximum droplet width value, which is a case where the amount of change is negative. Therefore, the vibration control system changes the amplitude selection criterion to an amplitude with which the width of the constricted portion is 20% or more of the maximum droplet width value, for example. In the case where the amplitude is 0.4 among these three cases, the width of the constricted portion is 20% or more of the maximum droplet width value. Therefore, 0.4 may be determined to be the amplitude. In the cases with width change amounts, the case with 0.4, which is equal to or greater than a predetermined minus value, may be determined to be the amplitude.

Further, in a case where there is a plurality of selectable amplitudes, any one of these amplitudes may be selected as desired, but a smaller amplitude is preferably selected. If the ratio of the harmonic amplitude to the fundamental wave amplitude becomes too high, the droplet shape is distorted and becomes unstable in some cases. Therefore, the vibration control system may select a smaller amplitude from a plurality of selectable amplitudes, or, for example, may select the smallest amplitude.

That is, according to the present disclosure, the vibration control system may determine a smaller amplitude, particularly the smallest amplitude, to be the amplitude of the harmonic from among a plurality of selectable amplitudes identified on the basis of the width of the liquid as described above.

In one embodiment, after step S107, the vibration control system carries out steps S108 to S110 described below.

In another embodiment, in response to the determination of the amplitude in step S107, the vibration control system may end the waveform parameter setting process. That is, in response to completion of the process in step S107, the vibration control system may determine the phase determined in step S105 and the amplitude determined in step S107 to be the phase and the amplitude of the harmonic.

### (Step S108)

In step S108, the vibration control system (particularly, the information processing unit) determines whether the shape of a droplet that is formed in a case where the phase determined in step S105 and the amplitude determined in step S107 are adopted as the waveform parameters of the harmonic is an appropriate shape.

The determination may be performed by comparing the shape of the droplet with a preferred predetermined droplet shape. In a case where the shape is the same as or similar to the predetermined droplet shape, the shape may be determined to be an appropriate shape. In a case where the shape is not similar to the predetermined droplet shape, the shape may be determined not to be an appropriate shape.

Alternatively, the determination may be performed on the basis of the size of a main droplet and/or a satellite droplet that is formed. When the sizes of these droplets fall within a predetermined numerical range, the droplets may be determined to be in an appropriate shape. When the sizes fall outside the predetermined numerical range, the droplets may be determined not to be in an appropriate shape.

If the droplets are determined not to be in an appropriate shape, the vibration control system advances the process to step S109.

If the droplets are determined to be in an appropriate shape, the vibration control system advances the process to step S110.

Alternatively, in step S108, a check may be made to determine whether the amplitude has been determined in step S107.

If the amplitude has not been determined (which is a case where there are no selectable amplitudes), the vibration control system advances the process to step S109.

If the amplitude has been determined, the vibration control system advances the process to step S110.

### (Step S109)

In step S109, the vibration control system (particularly, the information processing unit) performs a phase change process.

For example, in a case where the phase of the Fast satellite that is the satellite droplet to be absorbed by the main droplet at the earliest timing has been determined to be the phase of the harmonic in step S105, the vibration control system changes the phase of the harmonic to the phase of the Slow satellite that is the satellite droplet to be absorbed by the main droplet at the earliest timing.

Alternatively, in a case where the phase of the Slow satellite that is the satellite droplet to be absorbed by the main droplet at the earliest timing has been determined to be the phase of the harmonic in step S105, the vibration control system changes the phase of the harmonic to the phase of the Fast satellite that is the satellite droplet to be absorbed by the main droplet at the earliest timing.

The vibration control system may perform the phase change process in this manner.

Note that, in a case where only the phase of the Fast satellite that is the satellite droplet to be absorbed by the main droplet at the earliest timing has been identified in step S105, the vibration control system may identify the phase of the Slow satellite that is the satellite droplet to be absorbed by the main droplet at the earliest timing, and change the phase of the harmonic to the identified phase, for example.

Alternatively, in a case where only the phase of the Slow satellite that is the satellite droplet to be absorbed by the main droplet at the earliest timing has been identified in step S105, the vibration control system may identify the phase of the Fast satellite that is the satellite droplet to be absorbed by the main droplet at the earliest timing, and change the phase of the harmonic to the identified phase, for example.

That is, the vibration control system may again perform a process similar to step S105, and then change the phase of the harmonic to another phase identified by the process.

### (Step S110)

In step S110, the vibration control system (particularly, the information processing unit) performs a final adjustment process for the waveform parameters of the harmonic.

For example, the vibration control system adopts the amplitude determined in step S107 as the amplitude of the harmonic, and again performs a process similar to step S102. Note that the change in phase in step S110 may be changed more finely than the change in phase in step S102. In step S110, a process similar to step S102 is performed while changing the phase more finely, and droplet images are acquired.

In a case where a droplet image in which it is observed that the timing of absorption of a satellite droplet into the main droplet is earlier than that in case with the phase determined in step S105 is identified among the acquired droplet images, the vibration control system determines the phase corresponding to the droplet image and the amplitude determined in step S107 to be the phase and the amplitude of the harmonic.

In a case where a droplet image in which it is observed that the timing of absorption of a satellite droplet into the main droplet is earlier than that in case with the phase determined in step S105 is not identified among the acquired droplet images, the vibration control system determines the phase determined in step S105 and the amplitude determined in step S107 to be the phase and the amplitude of the harmonic.

### (Step S111)

In step S111, the flow cytometer 1 (particularly, the vibration control system 100) ends the waveform parameter setting process. After completion of the process, the flow cytometer 1 can perform a biological sample analysis process with the determined phase and amplitude of the harmonic. In the analysis process, the vibration control system of the flow cytometer 1 may vibrate the vibration element with a signal having a superimposed waveform in which a harmonic having the phase and the amplitude determined in the waveform parameter setting process is superimposed on the fundamental wave adopted in the setting process, to form droplets.

### (4) Feedback Control

In the description below, the flow cytometer 1 according to the present disclosure may perform a feedback control process to stabilize the shape of the droplets to be formed by driving the vibration element.

The feedback control process may be performed during the waveform parameter setting process described above.

For example, the feedback control process may be performed in the step (step S102 described above) of acquiring a droplet image in each phase while changing the phase of the harmonic in the waveform parameter setting process. That is, the control system may change the phase of the harmonic so that the position at which a droplet is separated from the liquid column, and the distance between the position and the separated droplet are maintained.

Also, the feedback control process may be performed in the step (step S106 described above) of acquiring a droplet image with each amplitude while changing the amplitude of the harmonic in the waveform parameter setting process. That is, the control system may change the phase of the harmonic so that the position at which a droplet is separated from the liquid column, and the distance between the position and the separated droplet are maintained.

Also, the feedback control process may be performed as an analysis process by the flow cytometer 1 is started. For example, the feedback control process may be started after the waveform parameter setting process described above.

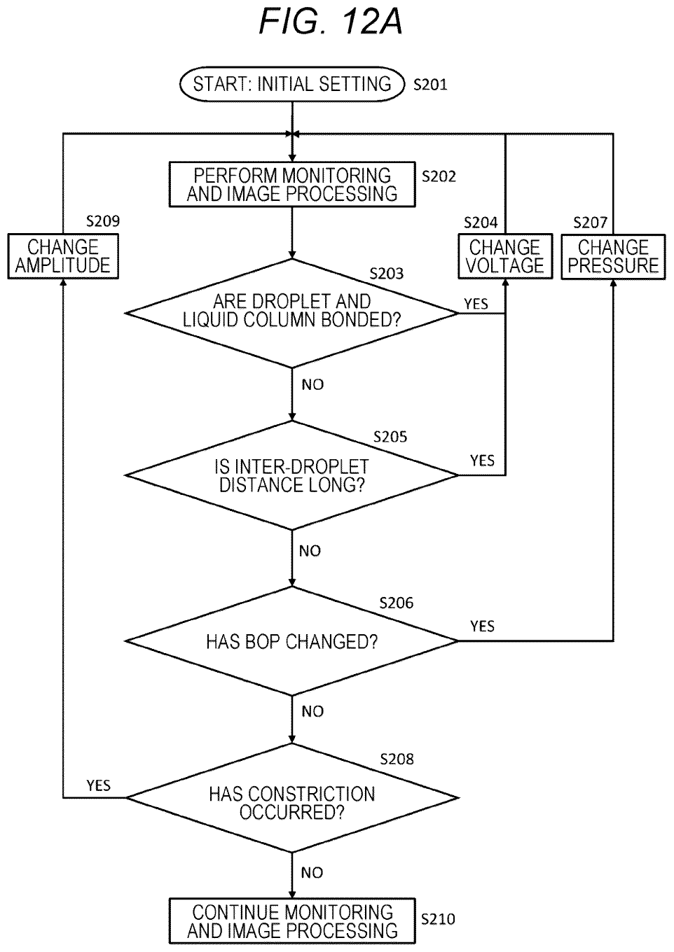

The feedback control process is described below with reference to FIG. 12A. The drawing is an example of a flowchart of the feedback control process.

In step S201, the flow cytometer 1 (particularly, the vibration control system) starts the feedback control process. At the start, the vibration control system of the flow cytometer 1 may adjust the phase of the strobe. The phase adjustment may be performed on the basis of the phase of the superimposed waveform of the signal to be applied to the vibration element (particularly, the phase of the fundamental wave). For example, the phase adjustment may be performed on the basis of the phase of the fundamental wave so as to be synchronized with the fundamental wave. The adjustment may be performed so that a droplet image captured by the droplet camera covers the state immediately after droplet separation.

In step S202, the vibration control system starts droplet imaging by the droplet camera. Also, at the start, image processing of the captured droplet image may be started. The imaging may be performed at predetermined intervals, or a moving image may be acquired.

In step S203, the vibration control system (particularly, the information processing unit) determines whether the BOP in the acquired droplet image has greatly changed. For example, a check may be made to determine whether the position of the BOP has changed so as to move upstream or downstream by an amount equivalent to one droplet or larger. A significant change in BOP is also referred to as a BOP jump. A BOP jump is now described with reference to FIG. 12B. As illustrated in the left portion of the drawing, a change in the position of the BOP so as to move upstream or downstream by an amount equivalent to one droplet or more is a BOP jump.

In response to a determination that a change has occurred, the vibration control system advances the process to step S204. In response to a determination that there are no changes, the flow cytometer 1 advances the process to step S205.

In step S204, the vibration control system (particularly, the information processing unit) changes the voltage to be applied to the vibration element.

For example, in a case where it is determined in step S203 that the position of the BOP has changed so as to move downstream, the vibration control system makes the voltage higher. Droplets are bonded to the liquid column due to external disturbance (the position of the BOP moves downstream), for example, and therefore, control may be performed to make the voltage of the vibration element higher at that time, to promote separation. Further, in a case where the position of the BOP changes so as to move upstream, the vibration control system may lower the voltage. For example, in a case where it is determined in step S205 that ΔBOP becomes shorter by a predetermined value or more, the flow cytometer 1 makes the voltage higher. Further, in a case where ΔBOP becomes longer by a predetermined value or more, the flow cytometer 1 lowers the voltage.

By the processes in steps S203 to S205, the BOP and ΔBOP can be maintained. Furthermore, the influence of minute external disturbance is observed from droplet images, and thus, the BOP and ΔBOP can be maintained with high accuracy. Further, the state (the BOP and ΔBOP, for example) immediately after a droplet is separated does not depend on the size, the shape, or the type of the droplet, a common determination criterion can be adopted in various analyses, and the same algorithm can be applied in various conditions.

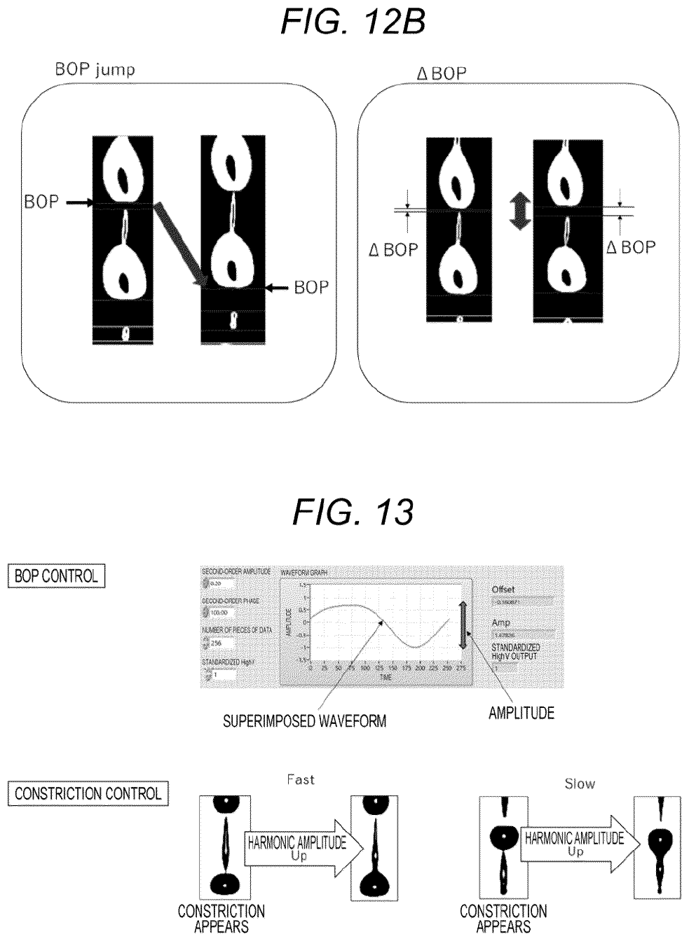

The voltage to be increased or decreased in step S204 may be controlled with the amplitude of the drive waveform of the voltage signal for driving the vibration element. That is, the increase or decrease in the voltage may be an increase or decrease in the amplitude of a combined waveform (superimposed waveform) of the fundamental wave and the harmonic (in the upper portion of FIG. 13). As described above, the vibration control system may adjust the BOP and/or ΔBOP by adjusting the amplitude of the superimposed waveform.

In step S205, the vibration control system (particularly, the information processing unit) determines whether ΔBOP in the acquired droplet image has changed. For example, a check may be made to determine whether ΔBOP has increased or decreased by a predetermined value or more. ΔBOP is now described with reference to FIG. 12B. As illustrated in the right portion of the drawing, ΔBOP may mean the distance between the liquid column and the liquid portion forming a satellite droplet. This distance may be counted with the number of pixels in the droplet image, for example. The predetermined value may be one pixel to ten pixels, or one pixel to five pixels, for example.

In response to a determination that a change has occurred, the vibration control system advances the process to step S204. In response to a determination that any change has not occurred, the vibration control system advances the process to step S206.

Step S206 is carried out in a case where it is determined that there is no change in both steps S203 and S205. In step S206, the vibration control system (particularly, the information processing unit) determines whether the BOP in the acquired droplet image has changed. This change may be a smaller change than the change in step S203.

In response to a determination that a change has occurred, the vibration control system advances the process to step S207. In response to a determination that any change has not occurred, the vibration control system advances the process to step S208.

In step S207, the vibration control system (particularly, the information processing unit) changes the liquid feeding pressure for the liquid to be discharged from the orifice.

For example, in a case where the position of the BOP changes so as to move downstream, the vibration control system lowers the liquid feeding pressure. Further, in a case where the position of the BOP changes so as to move upstream, the vibration control system lowers the liquid feeding pressure.

For example, a change in temperature may change the viscosity of the liquid, which might result in a change in flow rate. Furthermore, even if a certain pressure is maintained, the flow rate might change due to mixing or generation of bubbles. Such external disturbance cannot be adjusted with the voltage of the vibration element, and therefore, may be controlled with the liquid feeding pressure.

In step S208, the vibration control system (particularly, the information processing unit) determines the presence or absence of a decrease in the liquid width at the above-described bonding portion between the liquid portion forming a satellite droplet and the portion forming its main droplet. For example, the vibration control system may determine whether constriction has appeared. For the determination, the liquid width described above in step 107 in (3) may be referred to.

In response to a determination that the liquid width at the bonding portion has decreased, the vibration control system advances the process to step S209. In response to a determination that the liquid width has not decreased, the flow cytometer 1 advances the process to step S210.

In step S209, the vibration control system (particularly, the information processing unit) causes the amplitude of the harmonic to rise. With this rise, as illustrated in the lower side in FIG. 13, the liquid width can be increased in both cases with a Fast satellite and a Slow satellite. That is, it is possible to reduce the constriction that has occurred.

As described above, the vibration control system may change the bonding state between the liquid portion forming a satellite droplet and the liquid portion forming its main droplet by adjusting the amplitude of the harmonic, and, in particular, may adjust the width of the liquid portions.

In step S210, the vibration control system may continue the droplet imaging by the droplet camera, and the processes described in steps S203 to S209 may be then repeated.

As described above, the vibration control system according to the present disclosure may perform such a feedback control process based on the BOP and/or ΔBOP. Thus, it is possible to cope with fluctuations in the droplet forming state due to external disturbance during analysis over a long time, for example.

### (5) Examples

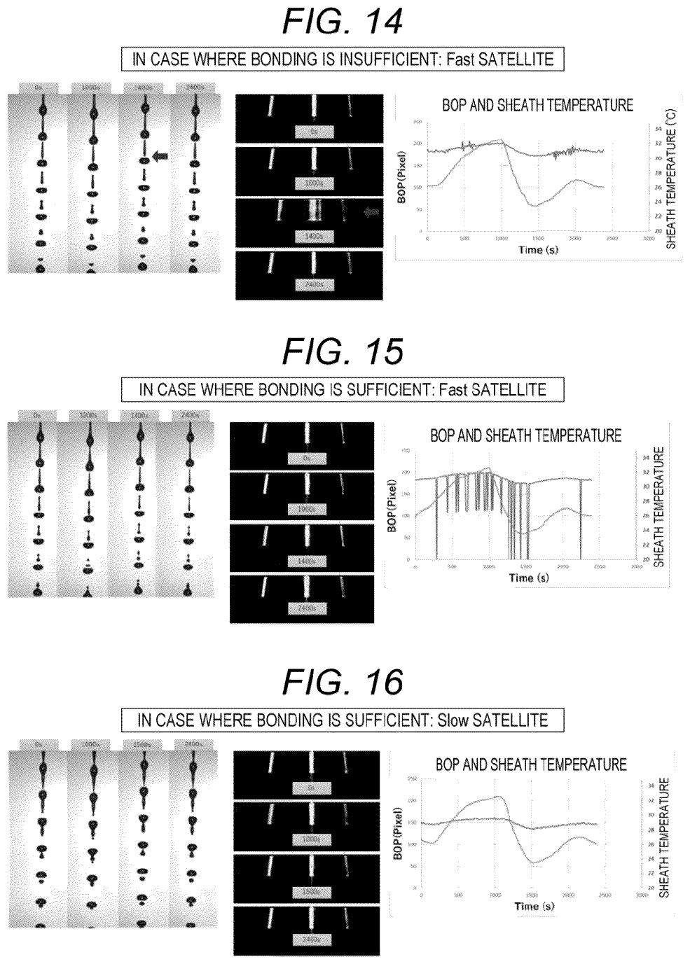

FIG. 14 illustrates an example in which the amplitude of the harmonic was set in the FAST satellite conditions under which the liquid portion forming a main droplet and the liquid portion forming a satellite droplet were separated from the liquid column while the bonding between these liquid portions was insufficient. When the temperature of the liquid to be sent was changed (the right portion in the drawing) in this situation, the timing of separation between the satellite droplet and the main droplet changed (the left portion in the drawing, in a case with 1400 s). At the same time, the amount of charge to be applied to the droplets also greatly changed, and a change was observed in the deflection distance of the side stream (in a case with 1400 s in the drawing).

FIG. 15 illustrates an example in which the amplitude of the harmonic was set in the FAST satellite conditions under which the liquid portion forming a main droplet and the liquid portion forming a satellite droplet were separated from the liquid column while the bonding between these liquid portions was sufficient. When the temperature of the liquid to be sent was changed (the right portion in the drawing) in this situation, the timing of separation between the satellite droplet and the main droplet was not greatly affected by the temperature change (the left portion in the drawing), unlike that in the case illustrated in FIG. 14. Furthermore, there was no change in the amount to charge to be applied to droplets, and there was no change in the deflection distance of the side stream (the center portion in the drawing).

FIG. 16 illustrates an example in which the amplitude of the harmonic was set in the SLOW satellite conditions under which the liquid portion forming a main droplet and the liquid portion forming a satellite droplet were separated from the liquid column while the bonding between these liquid portions was sufficient. When the temperature of the liquid to be sent was changed (the right portion in the drawing) in this situation, the timing of separation between the satellite droplet and the main droplet was not greatly affected by the temperature change (the left portion in the drawing), as in the case with the FAST conditions illustrated in FIG. 15. Furthermore, there was no change in the amount to charge to be applied to droplets, and there was no change in the deflection distance of the side stream (the center portion in the drawing).

### (6) Example Configuration of a Biological Sample Analyzer

The flow cytometer according to the present disclosure may be designed as a biological sample analyzer described below. The matters described below regarding the biological sample analyzer (a description regarding a biological sample, a flow channel, a light irradiation unit, a detection unit, an information processing unit, and a sorting unit) also apply to the flow cytometer according to the present disclosure. The present disclosure also provides a biological sample analyzer including the vibration control system described above.

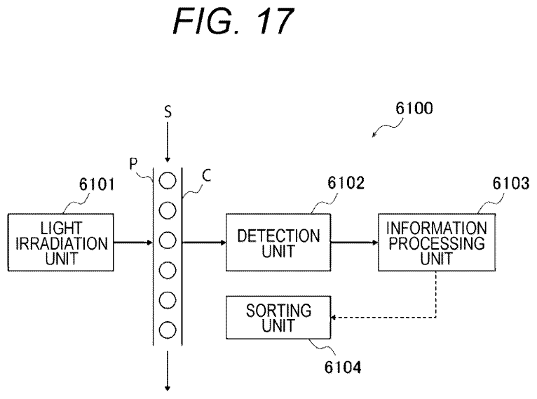

An example configuration of the biological sample analyzer is illustrated in FIG. 17. A biological sample analyzer 6100 illustrated in the drawing includes a light irradiation unit 6101 that irradiates a biological sample S flowing in a flow channel C with light, a detection unit 6102 that detects light generated by irradiating the biological sample S with light, and an information processing unit 6103 that processes information regarding the light detected by the detection unit. The biological sample analyzer 6100 is a flow cytometer or an imaging cytometer, for example. The biological sample analyzer 6100 may include a sorting unit 6104 that sorts out specific biological particles P in a biological sample. The biological sample analyzer 6100 including the sorting unit is a cell sorter, for example.

### (Biological Sample)

The biological sample S may be a liquid sample containing biological particles. The biological particles are cells or non-cellular biological particles, for example. The cells may be living cells, and more specific examples thereof include blood cells such as erythrocytes and leukocytes, and germ cells such as sperms and fertilized eggs. Also, the cells may be those directly collected from a sample such as whole blood, or may be cultured cells obtained after culturing. The non-cellular biological particles are extracellular vesicles, or particularly, exosomes and microvesicles, for example. The biological particles may be labeled with one or more labeling substances (such as a dye (particularly, a fluorescent dye) and a fluorochrome-labeled antibody). Note that particles other than biological particles may be analyzed by the biological sample analyzer of the present disclosure, and beads or the like may be analyzed for calibration or the like.

### (Flow Channel)

The flow channel C is designed so that a flow of the biological sample S is formed. In particular, the flow channel C may be designed so that a flow in which the biological particles contained in the biological sample are aligned substantially in one row is formed. The flow channel structure including the flow channel C may be designed so that a laminar flow is formed. In particular, the flow channel structure is designed so that a laminar flow in which the flow of the biological sample (a sample flow) is surrounded by the flow of a sheath liquid is formed. The design of the flow channel structure may be appropriately selected by a person skilled in the art, or a known one may be adopted. The flow channel C may be formed in a flow channel structure such as a microchip (a chip having a flow channel on the order of micrometers) or a flow cell. The width of the flow channel C is 1 mm or smaller, or particularly, may be not smaller than 10 μm and not greater than 1 mm. The flow channel C and the flow channel structure including the flow channel C may be made of a material such as plastic or glass.

The biological sample analyzer of the present disclosure is designed so that the biological sample flowing in the flow channel C, or particularly, the biological particles in the biological sample are irradiated with light from the light irradiation unit 6101. The biological sample analyzer of the present disclosure may be designed so that the irradiation point of light on the biological sample is located in the flow channel structure in which the flow channel C is formed, or may be designed so that the irradiation point is located outside the flow channel structure. An example of the former case may be a configuration in which the light is emitted onto the flow channel C in a microchip or a flow cell. In the latter case, the biological particles after exiting the flow channel structure (particularly, the nozzle portion thereof) may be irradiated with the light, and a flow cytometer of a jet-in-air type can be adopted, for example.

### (Light Irradiation Unit)

The light irradiation unit 6101 includes a light source unit that emits light, and a light guide optical system that guides the light to the irradiation point. The light source unit includes one or more light sources. The type of the light source(s) is a laser light source or an LED, for example. The wavelength of light to be emitted from each light source may be any wavelength of ultraviolet light, visible light, and infrared light. The light guide optical system includes optical components such as beam splitters, mirrors, or optical fibers, for example. The light guide optical system may also include a lens group for condensing light, and includes an objective lens, for example. There may be one or more irradiation points at which the biological sample and light intersect. The light irradiation unit 6101 may be designed to collect light emitted onto one irradiation point from one light source or different light sources.

### (Detection Unit)

The detection unit 6102 includes at least one photodetector that detects light generated by emitting light onto biological particles. The light to be detected may be fluorescence or scattered light (such as one or more of the following: forward scattered light, backscattered light, and side scattered light), for example. Each photodetector includes one or more light receiving elements, and has a light receiving element array, for example. Each photodetector may include one or more photomultiplier tubes (PMTs) and/or photodiodes such as APDs and MPPCs, as the light receiving elements. The photodetector includes a PMT array in which a plurality of PMTs is arranged in a one-dimensional direction, for example. The detection unit 6102 may also include an image sensor such as a CCD or a CMOS. With the image sensor, the detection unit 6102 can acquire an image (such as a bright-field image, a dark-field image, or a fluorescent image, for example) of biological particles.

The detection unit 6102 includes a detection optical system that causes light of a predetermined detection wavelength to reach the corresponding photodetector. The detection optical system includes a spectroscopic unit such as a prism or a diffraction grating, or a wavelength separation unit such as a dichroic mirror or an optical filter. The detection optical system is designed to disperse the light generated by light irradiation to biological particles, for example, and detect the dispersed light with a larger number of photodetectors than the number of fluorescent dyes with which the biological particles are labeled. A flow cytometer including such a detection optical system is called a spectral flow cytometer. Further, the detection optical system is designed to separate the light corresponding to the fluorescence wavelength band of a specific fluorescent dye from the light generated by the light irradiation to the biological particles, for example, and cause the corresponding photodetector to detect the separated light.

The detection unit 6102 may also include a signal processing unit that converts an electrical signal obtained by a photodetector into a digital signal. The signal processing unit may include an A/D converter as a device that performs the conversion. The digital signal obtained by the conversion performed by the signal processing unit can be transmitted to the information processing unit 6103. The digital signal can be handled as data related to light (hereinafter, also referred to as “light data”) by the information processing unit 6103. The light data may be light data including fluorescence data, for example. More specifically, the light data may be data of light intensity, and the light intensity may be light intensity data of light including fluorescence (the light intensity data may include feature quantities such as area, height, and width).

### (Information Processing Unit)

The information processing unit 6103 includes a processing unit that performs processing of various kinds of data (light data, for example), and a storage unit that stores various kinds of data, for example. In a case where the processing unit acquires the light data corresponding to a fluorescent dye from the detection unit 6102, the processing unit can perform fluorescence leakage correction (a compensation process) on the light intensity data. In the case of a spectral flow cytometer, the processing unit also performs a fluorescence separation process on the light data, and acquires the light intensity data corresponding to the fluorescent dye. The fluorescence separation process may be performed by an unmixing method disclosed in JP 2011-232259 A, for example. In a case where the detection unit 6102 includes an image sensor, the processing unit may acquire morphological information about the biological particles, on the basis of an image acquired by the image sensor. The storage unit may be designed to be capable of storing the acquired light data. The storage unit may be designed to be capable of further storing spectral reference data to be used in the unmixing process.

In a case where the biological sample analyzer 6100 includes the sorting unit 6104 described later, the information processing unit 6103 can determine whether to sort the biological particles, on the basis of the light data and/or the morphological information. The information processing unit 6103 then controls the sorting unit 6104 on the basis of the result of the determination, and the biological particles can be sorted by the sorting unit 6104.

The information processing unit 6103 may be designed to be capable of outputting various kinds of data (such as light data and images, for example). For example, the information processing unit 6103 can output various kinds of data (such as a two-dimensional plot or a spectrum plot, for example) generated on the basis of the light data. The information processing unit 6103 may also be designed to be capable of accepting inputs of various kinds of data, and accepts a gating process on a plot by a user, for example. The information processing unit 6103 may include an output unit (such as a display, for example) or an input unit (such as a keyboard, for example) for performing the output or the input.

The information processing unit 6103 may be designed as a general-purpose computer, and may be designed as an information processing device that includes a CPU, a RAM, and a ROM, for example. The information processing unit 6103 may be included in the housing in which the light irradiation unit 6101 and the detection unit 6102 are included, or may be located outside the housing. Further, the various processes or functions to be executed by the information processing unit 6103 may be realized by a server computer or a cloud connected via a network.

### (Sorting Unit)

The sorting unit 6104 performs sorting of biological particles, in accordance with the result of determination performed by the information processing unit 6103. The sorting method may be a method by which droplets containing biological particles are generated by vibration, electric charges are applied to the droplets to be sorted, and the traveling direction of the droplets is controlled by an electrode. The sorting method may be a method for sorting by controlling the traveling direction of biological particles in the flow channel structure. The flow channel structure has a control mechanism based on pressure (injection or suction) or electric charge, for example. An example of the flow channel structure may be a chip (the chip disclosed in JP 2020-76736 A, for example) that has a flow channel structure in which the flow channel C branches into a recovery flow channel and a waste liquid flow channel on the downstream side, and specific biological particles are collected in the recovery flow channel.

Furthermore, the biological sample analyzer described above may be designed as an information processing device according to the present disclosure. For example, the information processing unit 6103 may function as the information processing unit 103 according to the present disclosure, and may be designed to perform the process described above in (3-2) or (3-3), for example.

### 2. Second Embodiment (Waveform Parameter Setting Method)

The present disclosure also provides a method for setting waveform parameters of a signal for driving a droplet-generating vibration element of a flow cytometer. The vibration element may be driven with a signal having a waveform (also referred to as a superimposed waveform) obtained by superimposing a harmonic on the waveform of the fundamental frequency. In one embodiment, the setting method may include a setting process of setting waveform parameters, on the basis of a change caused in a satellite droplet by a change in the waveform parameters of the harmonic. The setting process may be performed as described above in 1. (particularly, (3) in 1.), for example. Also, the setting process may be performed by the flow cytometer (particularly, the vibration control system) described above in 1. (particularly, (2) in 1.). In this manner, the matters described above in 1. also apply to the setting method according to the present disclosure.

### 3. Third Embodiment (Program)

The present disclosure also provides a program for causing a flow cytometer (particularly, a vibration control system) to implement the waveform parameter setting method described above in 1, and 2. The setting method is as described above in 1, and 2., and the description therein also applies to the present embodiment. The program according to the present disclosure may be recorded in the recording medium described above, for example, or may be stored in the information processing unit or the storage unit described above.

Note that the present disclosure can also have configurations as described below.

[1]

A flow cytometer including
a vibration control system that controls vibration of a vibration element that generates a droplet,
in which the vibration control system is configured to drive the vibration element with a signal having a waveform in which a harmonic is superimposed on a waveform of a fundamental frequency, and
the vibration control system sets a waveform parameter, on the basis of a change caused in a satellite droplet by a change in the waveform parameter of the harmonic.
   [2]

The flow cytometer according to [1], in which the vibration control system sets a phase of the harmonic, an amplitude of the harmonic, or both a phase and an amplitude of the harmonic, on the basis of a satellite droplet in an image of a generated droplet.

[3]

The flow cytometer according to [1] or [2], in which the vibration control system sets a phase of the harmonic on the basis of a satellite droplet in an image of a generated droplet, and next sets an amplitude of the harmonic on the basis of a satellite droplet in an image of a droplet generated when a harmonic having the set phase is adopted.

[4]

The flow cytometer according to any one of [1] to [3], in which the vibration control system sets a phase of the harmonic to change a timing at which a satellite droplet is recovered into a main droplet to an earlier timing.

[5]

The flow cytometer according to any one of [1] to [4], in which the vibration control system sets a phase of the harmonic, on the basis of a change caused in a satellite droplet image by a change in phase of the harmonic.

[6]

The flow cytometer according to any one of [1] to [5], in which
the vibration control system
acquires a droplet image in each changed phase while changing the phase of the harmonic, and
determines the phase of the harmonic, on the basis of the acquired droplet images.
   [7]

The flow cytometer according to [6], in which the vibration control system changes the phase of the harmonic to maintain a position at which a droplet is separated from a liquid column, and a distance between the position and the separated droplet.

[8]

The flow cytometer according to [6] or [7], in which the vibration control system performs a classification process of classifying types of satellite droplets in each of the acquired droplet images, and a phase identification process of identifying an optimum phase on the basis of a classification result in the classification process.

[9]

The flow cytometer according to [8], in which, in the classification process, a satellite droplet is classified as a Fast satellite or a Slow satellite.

[10]

The flow cytometer according to any one of [1] to [9], in which the vibration control system sets an amplitude of the harmonic, to separate a liquid portion forming a satellite droplet and a liquid portion forming a main droplet from a liquid column while the liquid portions are bonded to each other.

[11]

The flow cytometer according to any one of [1] to [10], in which the vibration control system sets an amplitude of the harmonic, on the basis of a change caused in a satellite droplet image by a change in amplitude of the harmonic.

[12]

The flow cytometer according to any one of [1] to [11], in which
the vibration control system
acquires a droplet image with each changed amplitude while changing the amplitude of the harmonic, and
determines the amplitude of the harmonic, on the basis of the acquired droplet images.
   [13]

The flow cytometer according to [12], in which the vibration control system changes the amplitude of the harmonic to maintain a position at which a droplet is separated from a liquid column, and a distance between the position and the separated droplet.

[14]

The flow cytometer according to any one of [1] to [12], in which the vibration control system determines an amplitude of the harmonic, to separate a liquid portion forming a satellite droplet and a liquid portion forming a main droplet from a liquid column while the liquid portions are bonded to each other.

[15]

The flow cytometer according to any one of [12] to [14], in which the vibration control system determines an amplitude of the harmonic, on the basis of a change in a state of bonding between a liquid portion forming a satellite droplet and a liquid portion forming a main droplet.

[16]

The flow cytometer according to any one of [12] to [15], in which the vibration control system determines an amplitude of the harmonic, on the basis of a width of a bonding portion between a liquid portion forming a satellite droplet and a liquid portion forming a main droplet.

[17]

The flow cytometer according to any one of [1] to [16], in which the vibration control system is configured to adjust a position at which a droplet is separated from a liquid column, and/or a distance between the position and the separated droplet.

[18]

The flow cytometer according to [17], in which the vibration control system adjusts an amplitude of the superimposed waveform, to adjust the position at which the droplet is separated from the liquid column and/or the distance between the position and the separated droplet.

[19]

The flow cytometer according to any one of [1] to [18], in which the vibration control system adjusts an amplitude of the harmonic, to adjust widths of a liquid portion forming a satellite droplet and a liquid portion forming a main droplet.

[20]

A method for setting a waveform parameter of a signal for driving a droplet-generating vibration element of a flow cytometer, the method including
a setting process of setting the waveform parameter of the signal for driving the droplet-generating vibration element,
in which the signal is a signal having a waveform in which a harmonic is superimposed on a waveform of a fundamental frequency, and
the setting process is performed on the basis of a change caused in a satellite droplet by a change in a waveform parameter of the harmonic.

### REFERENCE SIGNS LIST

1 Flow cytometer
100 Vibration control system
101 Vibration element
102 Imaging unit
103 Information processing unit

1. A flow cytometer comprising
a vibration control system that controls vibration of a vibration element that generates a droplet,
wherein the vibration control system is configured to drive the vibration element with a signal having a waveform in which a harmonic is superimposed on a waveform of a fundamental frequency, and
the vibration control system sets a waveform parameter, on a basis of a change caused in a satellite droplet by a change in the waveform parameter of the harmonic.

2. The flow cytometer according to claim 1, wherein the vibration control system sets a phase of the harmonic, an amplitude of the harmonic, or both a phase and an amplitude of the harmonic, on a basis of a satellite droplet in an image of a generated droplet.

3. The flow cytometer according to claim 1, wherein the vibration control system sets a phase of the harmonic on a basis of a satellite droplet in an image of a generated droplet, and next sets an amplitude of the harmonic on a basis of a satellite droplet in an image of a droplet generated when a harmonic having the set phase is adopted.

4. The flow cytometer according to claim 1, wherein the vibration control system sets a phase of the harmonic to change a timing at which a satellite droplet is recovered into a main droplet to an earlier timing.

5. The flow cytometer according to claim 1, wherein the vibration control system sets a phase of the harmonic, on a basis of a change caused in a satellite droplet image by a change in the phase of the harmonic.

6. The flow cytometer according to claim 1, wherein
the vibration control system
acquires a droplet image in each changed phase while changing the phase of the harmonic, and
determines the phase of the harmonic, on a basis of the acquired droplet images.

7. The flow cytometer according to claim 6, wherein the vibration control system changes the phase of the harmonic to maintain a position at which a droplet is separated from a liquid column and a distance between the position and the separated droplet.

8. The flow cytometer according to claim 6, in which the vibration control system performs a classification process of classifying types of satellite droplets in each of the acquired droplet images, and a phase identification process of identifying an optimum phase on a basis of a classification result in the classification process.

9. The flow cytometer according to claim 8, wherein, in the classification process, a satellite droplet is classified as a Fast satellite or a Slow satellite.

10. The flow cytometer according to claim 1, wherein the vibration control system sets an amplitude of the harmonic, to separate a liquid portion forming a satellite droplet and a liquid portion forming a main droplet from a liquid column while the liquid portions are bonded to each other.

11. The flow cytometer according to claim 1, wherein the vibration control system determines an amplitude of the harmonic, on a basis of a change caused in a satellite droplet image by a change in the amplitude of the harmonic.

12. The flow cytometer according to claim 1, wherein
the vibration control system
acquires a droplet image with each changed amplitude while changing the amplitude of the harmonic, and
determines the amplitude of the harmonic, on a basis of the acquired droplet images.

13. The flow cytometer according to claim 12, wherein the vibration control system changes the amplitude of the harmonic to maintain a position at which a droplet is separated from a liquid column and a distance between the position and the separated droplet.

14. The flow cytometer according to claim 12, wherein the vibration control system determines an amplitude of the harmonic, to separate a liquid portion forming a satellite droplet and a liquid portion forming a main droplet from a liquid column while the liquid portions are bonded to each other.

15. The flow cytometer according to claim 12, wherein the vibration control system determines an amplitude of the harmonic, on a basis of a change in a state of bonding between a liquid portion forming a satellite droplet and a liquid portion forming a main droplet.

16. The flow cytometer according to claim 12, wherein the vibration control system determines an amplitude of the harmonic, on a basis of a width of a bonding portion between a liquid portion forming a satellite droplet and a liquid portion forming a main droplet.

17. The flow cytometer according to claim 1, wherein the vibration control system is configured to adjust a position at which a droplet is separated from a liquid column, and/or a distance between the position and the separated droplet.

18. The flow cytometer according to claim 17, wherein the vibration control system adjusts an amplitude of the superimposed waveform, to adjust the position at which the droplet is separated from the liquid column and/or the distance between the position and the separated droplet.

19. The flow cytometer according to claim 1, wherein the vibration control system adjusts an amplitude of the harmonic, to adjust widths of a liquid portion forming a satellite droplet and a liquid portion forming a main droplet.

20. A method for setting a waveform parameter of a signal for driving a droplet-generating vibration element of a flow cytometer, the method comprising
a setting process of setting the waveform parameter of the signal for driving the droplet-generating vibration element,
wherein the signal is a signal having a waveform in which a harmonic is superimposed on a waveform of a fundamental frequency, and
the setting process is performed on a basis of a change caused in a satellite droplet by a change in a waveform parameter of the harmonic.
